SEQUENCE LISTING (31 sequences)

SEQ ID NO: 1 - protein, 121 aa, Homo sapiens
  GS2
QVKLEESGGGLVQAGGSLKLSCAASGSTFSITTMGWYRQAPGKQRELVAGIIVVSDPVRPSINYADFVDGRFSVSRDNIENTLYLQMNSLKPEDTAVYYCAAVTSDHRGYWGQGTQVTVSS

SEQ ID NO: 2 - protein, 129 aa, Homo sapiens
  GS4
QVKLEESGGGSVEAGGSLRLSCAASGDITGDNCMAWFRQAPGKEREGVAVIYTATGSTYYTESVEGRFILSQDNAKNTVYLQMDSLDPEDTAIYYCARPTNNRYPWGGCPLYEDAYNYWGQGTQVTVSS

SEQ ID NO: 3 - protein, 129 aa, Homo sapiens
  GS5
QVKLEESGGGSVQAGGSLRLSCAASGDITGDNCMAWFRQAPGKEREGVAVIHTATDSTYYTESVEGRFILSQDNAKNTVYLQMDSLDPEDTAIYYCARPTNNRYPWGGCPLYEDAYNYWGQGTQVTVSS

SEQ ID NO: 4 - protein, 8 aa, Homo sapiens
  CDR1 of GS2
GSTFSITT

SEQ ID NO: 5 - protein, 7 aa, Homo sapiens
  CDR2 of GS2
IIVVSDP

SEQ ID NO: 6 - protein, 8 aa, Homo sapiens
  CDR3 of GS2
VTSDHRGY

SEQ ID NO: 7 - protein, 8 aa, Homo sapiens
  CDR1 of GS4 or GS5
GDITGDNC

SEQ ID NO: 8 - protein, 7 aa, Homo sapiens
  CDR2 of GS4
IYTATGS

SEQ ID NO: 9 - protein, 7 aa, Homo sapiens
  CDR2 of GS5
IHTATDS

SEQ ID NO: 10 - protein, 20 aa, Homo sapiens
  CDR3 of GS4 or GS5
PTNNRYPWGGCPLYEDAYNY

SEQ ID NO: 11 - protein, 140 aa, Homo sapiens
  HD13-H, heavy chain
PLLFTPVAKAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCTTMGNGNYWGQGTLVTVSSASTKGPSVFPLAPSS

SEQ ID NO: 12 - protein, 160 aa, Homo sapiens
  HD21-H, heavy chain
ENKVKQSTIALALLPLLFTPVAKAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREGSSGWYGLRDAFDIWGQGTMVTVSSASTKGPSVFPLA

SEQ ID NO: 13 - protein, 140 aa, Homo sapiens
  HD44-H, heavy chain
ALLPLLFTPVAKAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDAVYYDSSGHFYFDYWGQGTLVTVSSAST

SEQ ID NO: 14 - protein, 140 aa, Homo sapiens
  HD58-H, heavy chain
LLFTPVAKAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCATSSGIFYYYYYGMDVWGQGTTVTVSSASTKGPSV

SEQ ID NO: 15 - protein, 160 aa, Homo sapiens
  HD61-H, heavy chain
HGENKVKQSTIALALLPLLFTPVAKAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCAKFSNDVYYYYGMDVWGQGTMVTVSSASTKGPSVFPLA

SEQ ID NO: 16 - protein, 140 aa, Homo sapiens
  Consensus sequence of heavy chains
ALLPLLFTPVAKAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSYYGDVWGQGTLVTVSSASTKGPSVFPLA

SEQ ID NO: 17 - protein, 140 aa, Homo sapiens
  HD13-L, light chain
PTAAAGLLLLAAQPAMADIQLTQSPSTLSASVGDRVTITCRASQSIDNWLAWYQQKPGKVPKLLIYKASTLESGVPSRFSGSGSGTDFTLTISSLQPEDFATYYCQQSNSFPLTFGGGTKVEIKRTVAAPSVFIFPPSDE

SEQ ID NO: 18 - protein, 140 aa, Homo sapiens
  HD21-L, light chain
TAAAGLLLLAAQPAMAEIVLTQSPDTLSLSPGQRATLACRASETVSVNYLAWYQQTPGQAPRLLIYGASKRATGIPDRFSGSGSGTDFSLTISRLEPEDFVVYYCQQYAASPYTFGQGTKVEIKRTVAAPSVFIFPPSDE

SEQ ID NO: 19 - protein, 140 aa, Homo sapiens
  HD44-L, light chain
PTAAAGLLLLAAQPAMAVIWMTQSPDSLAVSLGERATINCKSSQSVLYSSNNKNYLVWYQQKVGQPPKPLISWASTRESGVPDRFSGSGSGTNFTLTVSSLQAEDVAVYYCLQYYRFPYTFGQGTKLEIKRTVAAPSVFI

SEQ ID NO: 20 - protein, 140 aa, Homo sapiens
  HD58-L, light chain
TAAAGLLLLAAQPAMAEIVMTQSPSSVSASVGDRVTITCRASHDISTWLAWYQQKPGKAPKLLIYKASSLESGVPFRFSGSGSGTEFTLTISSLQPADFATYYCQQAHSFPLTFGGGTKVEIKRTVAAPSVFIFPPSDEQ

SEQ ID NO: 21 - protein, 140 aa, Homo sapiens
  HD61-L, light chain
PTAAAGLLLLAAQPAMADIVMTQSPGTLSLSPGERATLSCRASESVSSSYLAWYQQKPGQAPRLLIYGASTRATGIPARFSGSGSGTDFTLTVSSLQPEDFGTYYCQQLYTYPITFGQGTRLEIKRTVAAPSVFIFPPSD

SEQ ID NO: 22 - protein, 135 aa, Homo sapiens
  Consensus sequence of light chains
PTAAAGLLLLAAQPAMADIVMTQSPTLSLSVGDRATITCRASSVSYLAWYQQKPGQAPKLLIYASTRESGVPRFSGSGSGTDFTLTISSLQPEDFATYYCQQYSFPLTFGQGTKVEIKRTVAAPSVFIFPPPSDE

SEQ ID NO: 23 - DNA, 21 bp, Homo sapiens
  hPROCR-shRNA-1
gcagcagctcaatgcctacaa

SEQ ID NO: 24 - DNA, 21 bp, Homo sapiens
  hPROCR-shRNA-3
tggcctccaaagacttcatat

SEQ ID NO: 25 - DNA, 20 bp, Homo sapiens
  hPROCR activation sgRNA
tcctgccggcgctgactcag

SEQ ID NO: 26 - DNA, 20 bp, Homo sapiens
  hPROCR suppression sgRNA
cagactccgcccctcccaga

SEQ ID NO: 27 - DNA, 21 bp, Homo sapiens
  shRNA sequences-PROCR
gcagcagctcaatgcctacaa

SEQ ID NO: 28 - DNA, 21 bp, Homo sapiens
  shRNA sequences-F2R
cccggtcatttcttctcagga

SEQ ID NO: 29 - DNA, 21 bp, Homo sapiens
  shRNA sequences-IGF-1R
gcggtgtccaataactacatt

SEQ ID NO: 30 - DNA, 21 bp, Homo sapiens
  shRNA sequences-EGFR
cgcaaagtgtgtaacggaata

SEQ ID NO: 31 - DNA, 20 bp, Homo sapiens
  sgRNAs sequence for PROCR activation
tcctgccggcgctgactcag

(19) United States
(12) Patent Application Publication
(10) Pub. No.: US 2019/0045758 A1
(43) Pub. Date: Feb. 14, 2019

(54) Biomarker and Therapeutic Target for Triple Negative Breast Cancer

(71) Applicant: Shanghai Institutes for Biological Sciences, Chinese Academy of Sciences, Shanghai (CN)

(72) Inventors: Yi Zeng, Shanghai (CN); Daisong Wang, Shanghai (CN)

(21) Appl. No.: 15/504,748

(22) PCT Filed: Aug. 19, 2015
(86) PCT No.: PCT/CN2015/087555

§ 371 Date: Feb. 17, 2017

(30) Foreign Application Priority Data
Aug. 20, 2014 (CN) 2014 10413021.6
Jun. 9, 2015 (CN) 2015 103 13730.1

(51) Int. Cl. A01K 67/027; A61K 48/00; C07K 16/30; C12N 15/11; A61P 35/00

(52) U.S. Cl. CPC A01K 67/0275; A61K 48/00; C12Q 1/6886; C12N 15/11; A61P 35/00; C07K 16/3015

(57) ABSTRACT

Provided herein are methods for diagnosing and/or treating triple negative breast cancers (TNBC), as well as compositions and kits that can be used in such methods. In one aspect, protein C receptor (PROCR) can be used in the diagnosis and/or treatment of TNBC, wherein the PROCR is selected from Procr gene, Procr mRNA and/or PROCR protein. The TNBC treatment can include one or more of: (i) RCR-252 antibody, or antigen binding fragment thereof; (ii) an isolated anti-PROCR antibody or antigen binding fragment thereof wherein the antibody cross-competes for binding to PROCR with RCR-252; (iii) soluble PROCR fragment; (iv) the interfering RNA designed to target Procr mRNA, and (v) CRISPR/Cas9 designed to target Procr gene.

### CROSS REFERENCE TO RELATED APPLICATIONS

This application claims priority to and the benefit of Chinese Patent Application Nos. 201410413021.6 filed on Aug. 20, 2014 and 201510313730.1 filed Jun. 9, 2015, the entire disclosures of which applications are incorporated herein by reference.

### FIELD

Provided herein are methods for diagnosing and/or treating triple negative breast cancers (e.g., tumors), as well as compositions and kits that can be used in such methods.

### BACKGROUND

In women, breast cancer is among the most common cancers and is the fifth most common cause of cancer deaths. Due to the heterogeneity of breast cancers, 10-year progression free survival can vary widely with stage and type, from 98% to 10%. Different forms of breast cancers can have remarkably different biological characteristics and clinical behavior. Thus, classification of a patient's breast cancer has become a critical component for determining a treatment regimen. For example, along with classification of histological type and grade, breast cancers now are routinely evaluated for expression of hormone receptors (estrogen receptor (ER) and progesterone receptor (PR)) and for expression of HER2 (ErbB2), since a number of treatment modalities are currently available that target hormone receptors or the HER2 receptor. ER and PR are both nuclear receptors (they are predominantly located at cell nuclei, although they can also be found at the cell membrane) and small molecular inhibitors that target ER and/or PR have been developed. HER2, or human epidermal growth factor receptor type 2, is a receptor normally located on the cell surface and antibodies that target HER2 have been developed as therapeutics. HER2 is the only member of the EGFR family (which also includes HER1 (EGFR), HER3 (ErbB3) and HER4 (ErbB4) that is not capable of binding to an activating ligand on its own. Thus HER2 is only functional as a receptor when incorporated into a heterodimeric receptor complex with another EGFR family member, such as HER3. Cancers classified as expressing the estrogen receptor (estrogen receptor positive, or ER⁺ tumors) may be treated with an ER antagonist such as tamoxifen. Similarly, breast cancers classified as expressing high levels the HER2 receptor may be treated with an anti-HER2 antibody, such as trastuzumab, or with a HER2-active receptor tyrosine kinase inhibitor such as lapatinib.

Triple negative breast cancer (TNBC) is a term used to designate a well-defined clinically relevant subtype of breast carcinomas that account for approximately 15% of all breast cancer cases. TN tumors score negative (i.e., using conventional histopathology methods and criteria) for expression of ER and PR and do not express amplified levels of HER2 (i.e., they are ER⁻, PR⁻, HER2⁻). TNBC comprises primarily, but not exclusively, a molecularly and histopathologically distinct subtype of breast cancer known as the basal-like (BL) subtype. The BL subtype also is characterized by the expression of cytokeratins (e.g., CK, CK5/6, CK14, CK17) and other proteins found in normal basal/myoepithelial cells of the breast. However, in addition to the BL subtype, certain other types of breast cancers, including some “normal breast-like”, metaplastic carcinomas, medullary carcinomas and salivary gland-like tumors can also exhibit the triple negative (TN) phenotype. Furthermore, TNBC occurs more frequently in the presence of BRCA1 mutations and in pre-menopausal females of African-American or Hispanic descent. TN tumors typically display very aggressive behavior, with shorter post-relapse survival and poor overall survival rates relative to other breast cancer types.

Given the lack of expression of hormone receptors or of significant amounts of HER2 in TNBC cells, treatment options have been very limited as the tumors are not responsive to treatments that target ER (e.g., tamoxifen, aromatase inhibitors) or HER2 (e.g., trastuzumab). Instead these tumors are treated with conventional neoadjuvant and adjuvant chemotherapy regimens, which have limited efficacy and many cytotoxic side effects. Furthermore, such chemotherapy regimens can lead to drug resistance in tumors, and the risk of recurrence of disease in TNBC is higher within the first three years of treatment than for other types of breast cancers.

A summary of currently available targeted treatments for the 4 types of breast cancers is shown below. Clearly, there is a major need to better understand the molecular basis, in particular specific biomarkers, of TNBC and to develop effective treatments for this aggressive type of breast cancer.

| | | | |
|---|---|---|---|
| Luminal A | Luminal B | Her2 | TNBC |
| (ER+/PR+, | (ER+/PR+, | (ER−, PR−, | (ER−, PR−, |
| Her2−) | Her2+) | Her2+) | Her2−) |

| | | |
|---|---|---|
| tamoxifen | Trastuzumab (Herceptin) | None |
| raloxifene (Evista) | Pertuzumab (Perjeta) | Available |
| toremifene (Fareston) | Ado-trastuzumab | |
| anastrozole (Arimidex) | emtansine (Kadcyla) | |
| letrozole (Femara) | Lapatinib (Tykerb) | |
| exemestane (Aromasin) | Bevacizumab (Avastin) | |
| fulvestrant (Faslodex) | | |

### SUMMARY

Provided herein are methods for diagnosing and/or treating triple negative breast cancers (e.g., tumors), as well as pharmaceutical compositions that can be used in such methods. The methods and compositions are based, at least in part, on the discovery that Procr gene expression is surprisingly correlated with TNBC, and that a neutralizing antibody that blocks PROCR and PROC binding can suppress the growth of TNBC cells. In particular, administration of anti-PROCR antibody is demonstrated to suppress the growth of TNBC cells in vitro and in vivo, as well as metastasis and epithelial-mesenchymal transition (EMT) of TNBC cells in vivo.

In one aspect, provided herein is protein C receptor (PROCR) for use in the diagnosis and/or treatment of triple negative breast cancer (TNBC) or a subgroup (PROCR-positive group) within TNBC, wherein the PROCR is selected from Procr gene, Procr mRNA and/or PROCR protein. In some embodiments, an elevated expression level compared to non-TNBC control indicates the presence of TNBC. The elevated expression level can be detected by an amount of PROCR mRNA and/or protein, which can be, for example, more than 50%, more than 100%, more than 150%, more than 200%, more than 250%, more than 300% higher than the non-TNBC control, or any number between 50%-500%, inclusive, or more. mRNA level can be detected by, for example, Northern blot or reverse transcription qPCR using probes or primers specifically designed to target Procr. Protein level can be detected by, for example, Western blot or immunostaining using an antibody against PROCR. Polyclonal and monoclonal antibodies can be generated using conventional methods known in the art. The antibody can be directly or indirectly labeled to facilitate detection in accordance with methods known in the art.

In some embodiments, decreasing PROCR level and/or activity can provide TNBC treatment. Decreasing PROCR level and/or activity can include one or more of: inhibiting Procr gene and/or mRNA stability and/or expression, reducing PROCR protein and/or neutralizing PROCR protein activity. In certain embodiments, the TNBC treatment can comprise a medicament selected from: (i) RCR-252 antibody, or antigen binding fragment thereof; (ii) an isolated anti-PROCR antibody or antigen binding fragment thereof wherein the antibody cross-competes for binding to PROCR with RCR-252; (iii) interfering RNA designed to target Procr mRNA, and (iv) CRISPR/Cas9 designed to target Procr gene.

In another aspect, provided herein is RCR-252 antibody, or antigen binding fragment thereof, for use in the diagnosis and/or treatment of triple negative breast cancer.

A further aspect relates to an isolated anti-PROCR antibody or antigen binding fragment thereof wherein the antibody cross-competes for binding to PROCR with RCR-252.

Another aspect relates to a kit for diagnosing TNBC, comprising one or more of: (a) primers and/or probes designed to detect Procr mRNA; (b) RCR-252 antibody, or antigen binding fragment thereof; and (c) an isolated anti-PROCR antibody or antigen binding fragment thereof wherein the antibody cross-competes for binding to PROCR with RCR-252. In some embodiments, the kit further includes instruction that when the amount of PROCR mRNA and/or protein in a sample is more than 50%, more than 100% or more than 200% or higher than a non-TNBC control, then the sample is from TNBC.

Also provided herein is a PROCR inhibitor for use in the preparation of a medicament for: (1) the treatment of triple negative breast cancer, (2) the inhibition of growth of TNBC cells, (3) the reduction of metastasis of TNBC cells, and/or (4) the inhibition of epithelial-mesenchymal transition (EMT) of TNBC cells. The PROCR inhibitor can be, in some embodiments, selected from the group consisting of: (i) RCR-252 antibody, or antigen binding fragment thereof; (ii) an isolated anti-PROCR antibody or antigen binding fragment thereof wherein the antibody cross-competes for binding to PROCR with RCR-252; (iii) soluble PROCR fragment (e.g., preferably comprising amino acids 1-210 or 18-210 of SEQ ID NO:2); (iv) the interfering RNA designed to target Procr mRNA, and (v) CRISPR/Cas9 designed to target Procr gene.

In a further aspect, provided herein is a pharmaceutical composition for treating triple negative breast cancer, comprising the PROCR inhibitor described herein and a pharmaceutically acceptable carrier.

Use of the PROCR inhibitor described herein for the manufacture of a medicament for the treatment of TNBC is also provided herein.

Another aspect relates to a method of suppressing growth, metastasis and/or EMT of a TNBC cell, comprising contacting the cell with an effective amount of the PROCR inhibitor disclosed herein.

Also provided herein is a method of identifying a PROCR inhibitor comprising: (a) providing a test agent to a plurality of PROCR-expressing TNBC cells, and (b) determining one or more of (1) PROCR expression level, (2) PROCR activity, and (3) survival and/or proliferation rate of the TNBC cells, wherein a decrease compared to a negative control not treated by the test agent indicates that the test agent is a PROCR inhibitor.

Another method of identifying a PROCR inhibitor comprises: (a) providing a test agent to a patient-derived xenograft, and (b) determining one or more of (1) PROCR expression level, (2) PROCR activity, and (3) tumor growth, metastasis and/or EMT in the xenograft, wherein a decrease compared to a negative control not treated by the test agent indicates that the test agent is a PROCR inhibitor.

A further method of identifying a PROCR inhibitor comprises: (a) providing a test agent, and (b) determining whether the test agent has one or more of the following characteristics: (i) binding to PROCR; (ii) interfering with or inhibiting binding of PROCR with protein C; (iii) cross-competing with RCR-252; (iv) interfering with or inhibiting binding of RCR-252 with PROCR; and/or (v) enhancing binding of RCR-252 with PROCR; wherein the test agent is a PROCR inhibitor if it has one or more of (i)-(v).

In any of the method of identifying a PROCR inhibitor above, the method can be performed in vitro or in vivo (e.g., in a TNBC xenograft model). In some embodiments, the test agent can be an antibody, a small molecule (e.g., a chemical), a peptide, a nucleic acid (e.g., shRNA, antisense RNA, siRNA), or a gene silencing tool (e.g., CRISPR/Cas9, TALENs, zinc finger nuclease). In some examples, the nucleic acid can be shRNA selected from one or more of SEQ ID NOS.: 19-22.

### BRIEF DESCRIPTION OF THE FIGURES

FIGS. 1a-1f: Procr⁺ Basal Cells Express Lower Levels of Basal Keratin.

a, b, Basal and luminal cells were FACS-isolated and analysed for Procr expression by qPCR. c, FACS analysis of Procr expression in 8-week-old CD1 mammary epithelial cells. d, Immunohistochemistry indicating the expression of Procr in a subpopulation of basal cells (arrows). Ninety-four per cent of Procr⁻ basal cells (n=206) expressed less K14 compared with the neighbouring basal cells (arrowheads). DAPI, 4′,6-diamidino-2-phenylindole. Scale bar, 20 μm. e, Expression of EMT-related genes in Procr⁺ basal cells. f, qPCR analysis of Procr⁺ basal cells and Procr⁻ basal cells. Quantification was performed on three independent experiments. E-Cad, E-cadherin. b, f, Data are presented as mean±standard deviation (s.d.). ***P<0.01.

FIGS. 2a-2e: Procr⁺ Cells Are Enriched For Mammary Stem Cells With Regenerative Capabilities.

a, Isolation of total basal, Procr⁺ basal and Procr⁻ basal populations. b, Colony-formation efficiency and the colony sizes in Matrigel culture. Scale bars, 20 μm. ***P<0.01. NS, not significant. See also FIG. 6a. c, d, Whole-mount and section images of an outgrowth derived from the transplantation of Procr⁺ basal cells in nulliparous and late pregnant mammary tissues. Scale bars, 2 mm in whole mount; 20 μm in section. e, Transplantation of sorted cells in limiting dilution. Data are pooled from four independent experiments. ***P<0.01.

FIGS. 3a-3d: Procr^{CreERT2-IRES-tdTomato} Knock-In Mouse Recapitulates the Procr Expression Pattern and Labelled Cell Behaviour.

a, Targeting strategy to generate the Procr^{CreERT2-IRES-tdTomato} knock-in (KI) mouse. See also FIG. 6a. b, Immunostaining analysis of the knock-in mammary sections. Scale bar, 20 μm. c, FACS analysis indicating that tdTomato⁺ cells are located in 3% of basal and 8% of stromal cells. n=4 mice. One of four similar experiments is shown. d, Colony formation of tdTomato⁺ and tdTomato⁻ basal cells in Matrigel culture. Scale bars, 20 μm. ***P<0.01.

FIGS. 4a-4p: Procr Labels Multipotent Adult Mammary Stem Cells.

a, Illustration of lineage tracing strategy. tdT, tdTomato. b, Experimental setup used in short-term tracing. c, d, FACS analysis indicating that GFP⁺ cells were restricted to the basal cells at 48 h after tamoxifen (TAM) administration. e, Whole-mount confocal microscopy showing an elongated GFP⁺ basal cell. f, Section imaging indicating the basal location of the GFP⁺ cell. g-k, FACS and imaging analysis of GFP⁺ cell distribution after 3 weeks of tracing. l-p, FACS and imaging analysis of GFP⁺ cells distribution at pregnant day 14.5 after 5 weeks of tracing. Scale bars, 20 μm. d, i, n, Data are presented as mean±s.d.n=3 mice.

FIGS. 5a-5g: No Procr⁺ Cells Are Found in Luminal Cells Throughout Postnatal Development.

a, b, Microarray of three-dimensional cultured basal cells in the presence of Wnt3A versus vehicle. 1 and 2 represent two independent experiments. See Methods for details. qPCR indicated that Procr is upregulated in basal cells cultured in the presence of Wnt3A compared with cells grown in the absence of Wnt3A. Data are pooled from three independent experiments. Data are presented as mean±s.d. ***P<0.01. c-g, The 4th inguinal mammary glands harvested from 2-week-old (c), 5-week-old (d), 8-week-old (e), pregnant day 14.5 (f) and 2 weeks post-weaning (g) CD1 mice were analysed by FACS. Procr⁺ cells were distributed in basal cells (ranging from 2.9% to 8.8%) and stromal fibroblasts (from 17.2% to 30.5%). No Procr⁺ cells were detected in luminal cells at any postnatal stage.

FIGS. 6a-6e: Generation of the Procr^{CreERT2-IRES-tdTomato} Knock-In Mouse.

a, Basal (Lin⁻ CD24⁺ CD29^{hi}), Procr⁺ CD24⁺ CD29^{hi} and Procr⁻ CD24⁺ CD29^{hi} cells were FACS-isolated and placed in Matrigel to assess the colony-formation ability. Data are pooled from four independent experiments. ***P<0.01. b, Targeting strategy to generate the Procr^{CreERT2-IRES-tdTomato/+} knock-in (KI) mouse. Designs of Southern blot probe and genotyping primers are as indicated. c, Southern blot analysis with a 5′ external probe of EcoRI-digested DNA from mouse embryonic stem cells, showing a 5.7 kb band in addition to the 7.7 kb wild-type (WT) band in clones that have undergone homologous recombination at the Procr locus. d, e, Embryos resulting from a cross of heterozygous male and female mice were dissected at E10.5 (d). Genotyping PCR indicated the proper distribution of wild type and heterozygotes as Mendel's law of segregation, and that homozygotes were lethal before this time point as the embryo had mostly been absorbed (d, e). One of three similar experiments is shown.

FIGS. 7a-7f: Procr⁺ cells and Lgr5⁺ cells are mutually exclusive populations in the mammary gland, while Procr⁺ cells and Axin2⁺ cells are largely non-overlapping in mammary basal cells.

a, Procr⁺ CD24⁺ CD29^{hi} and Procr⁻ CD24⁺ CD29^{hi} cells were FACS-isolated and analysed by qPCR. Procr⁻ cells expressed significantly lower levels of Lgr5 compared with Procr⁻ cells. Data are pooled from three independent experiments. ***P<0.01. b, Cells isolated from Lgr5-GFP mammary gland were analysed for the expression of GFP and Procr. 5.8% of basal cells (Lin⁻ CD24⁺ CD29^{hi}) were Lgr5-GFP⁺ cells, while 2.4% of basal cells were Procr⁺ cells. These two populations were not overlapped. c, Three basal subpopulations as indicated were FACS-isolated and cultured in Matrigel for colony formation. Only Procr⁺ Lgr5 cells formed colonies whereas Procr⁻Lgr5⁺ and Procr⁻ Lgr5⁻ cells could not. Data are presented as mean±s.d. Scale bars, 20 μm. ***P<0.01. d, Recipient fat pads were injected with freshly sorted basal subpopulation cells as indicated and harvested at 8 weeks after surgery. Procr⁻Lgr5⁻ cells efficiently formed new mammary glands (frequency 1/14.4). Comparably, Procr⁻Lgr5⁺ cells had significantly lower reconstitution efficiency (1/165.4). Procr⁻Lgr5⁻ cells were not able to reconstitute. e, Procr⁺CD24⁺CD29^{hi} and Procr⁻CD24⁻CD29^{hi} cells were FACS-isolated and analysed by qPCR. No significant difference in Axin2 level was detected in the two populations. Data are pooled from three independent experiments. ***P<0.01. f, Procr⁺CD24⁺CD29^{hi} and Procr⁻CD24⁺CD29^{hi} cells were isolated from Axin2-lacZ mammary gland and underwent X-gal staining. About 1.5% of Procr⁺CD24⁺CD29^{hi} cells were Axin2-lacZ⁺, while 6.0% of Procr⁻CD24⁺CD29^{hi} cells were Axin2-lacZ⁺. Data are pooled from two independent experiments (n=1,085 cells and n=1,103 cells).

FIGS. 8a-8h: Procr⁺ cells are located in mammary ducts and are proliferative cells.

a-e, The 4th inguinal mammary glands harvested from E18.5 (a), P1.5 (b), 5-week-old (c, d) and 8-week-old (e) Procr^{CreERT2-IRES-tdTomato/+} mice were analyzed by whole-mount confocal imaging. Individual tdTomato⁻ cells were dispersedly located in all stages of mammary ducts. tdTomato⁺ cells were not found in TEBs of 5-week-old glands (c). A minimum of 50 TEBs and 50 ducts were analysed. Scale bars, 100 μm. f-i, Analysis of proliferative cells in TEBs and ducts of Procr^{CreERT2-IRES-tdTomato} mice at 3 h after EdU injection. Five-week-old TEBs exhibited abundant EdU⁺ cells (green) but no Procr⁺ cells (red) (f). EdU⁺ Procr⁺ cells (yellow) were found in both 5-week-old ducts (g) and 8-week-old mammary gland (h). Quantification is shown in i. Scale bars, 20 μm.

FIGS. 9a-9f: Quantitative clonal analysis of Procr-labelled cells in mammary glands induced in puberty.

a-f, The number of basal and luminal cells in individual GFP⁺ clones were scored in Procr^{CreERT2/+}; R26^{mTmG/+} mammary glands after 3 weeks (a-c) or 6 weeks (d-f) induction. Basal cell numbers are shown along the y-axis, and luminal cell numbers are shown along the x-axis. Red shading indicates the relative frequency of certain clone composition, with deeper shading indicating higher frequency. d, Note that the deeper shading boxes shifted to the right in tracing experiments undertaken for a longer period. b, In clones after 3-week tracing, 72.4% were bi-lineage, 14.8% were solely basal cells derived from Procr⁺ cell division, 12.8% were single basal cells that had not divided. c, Among two-cell clones, 64.9% were composed of one basal cell and one luminal cell, while 35.1% consisted of two basal cells. d, e, In clones after 6-week tracing, the proportion of bi-lineage clone increased to 93%, while the percentage of the other two groups decreased to 3.9% and 3.1%. f, In two-cell clones, bi-lineage clones increased to 85.2%, while clones consisting of two basal cells decreased to 4.8%. n=4 mice for 3-week tracing and n=3 mice for 6-week tracing.

FIGS. 10a-10f: Procr⁺ cells are long-lived multipotent MaSCs retained beyond multiple rounds of pregnancy

a-d, Tamoxifen (TAM) was administered in 5-week-old Procr^{CreERT2/+}; R26^{mTmG/+} mice. Labelled cell contribution was analysed as illustrated in a. b-e, FACS analysis indicating that GFP⁺ cells are distributed in both basal and luminal layer in mid-2nd pregnancy (b, c) and mid-3rd pregnancy (d, e). f, Quantification of GFP⁺cells indicating no difference in the percentage of GFP⁺ basal cells between nulliparous mice and multiparous mice that have gone through three complete cycles of pregnancy and involution. n=3 mice. c, e, f, Data are presented as mean±s.d.

FIGS. 11a-11n: Procr labels multipotent mammary stem cells in mature adult mice.

a-g,Tamoxifen (TAM) was administered in 8-week old Procr^{CreERT2/+}; R26^{mTmG/+} mice. Labelled cell contribution was analysed after 3-week or 6-week induction. After 3 weeks, FACS analysis indicated that GFP⁺cells were distributed in both basal and luminal layers (b, c). Immunostaining in sections showed the clonal expansion of GFP⁺cells and confirmed their distribution in both basal and luminal layers. Basal cells were marked by K14, while cells apical to K14⁺ cells were luminal cells (arrow and arrowhead in d). Clonal analysis indicated that bi-lineage clones are the majority in all clones (74.2%) (e, f), and in two-cell clones (70.2%) (g). h-j, Clonal analysis of 6-week induction indicating that clone sizes are larger (red shaded boxes shifted to the right) (h), bi-lineage clone percentage has also increased to 94% in all clones (i) and to 89.5% in two-cell clones (j). k-n, Mammary glands were analysed at pregnant day 14.5 after tamoxifen administration at 8 weeks. GFP⁺cells were in both basal and luminal layers as indicated by FACS analysis (l, m). GFP⁺ cells contributed to alveologenesis by immunohistochemistry analysis in sections (n). n=3 mice for 3-week tracing and n=3 mice for 6-week tracing. Scale bars, 20 μm. c, m, Data are presented as mean±s.d.

FIGS. 12a-12n: Procr⁺ cells are multipotent MaSCs in embryonic or newborn mammary gland.

a-g, Tamoxifen (TAM) was administered in pregnant day 18.5 mothers bearing Procr^{CreERT2/+};R26^{mTmG/+} mice. Labelled cell contribution in the pups was analysed after 8-week induction (a). FACS analysis indicated that GFP⁺cells are distributed in both basal and luminal layers (b, c). Immunostaining in sections showed the clonal expansion of GFP⁺ cells and confirmed their distribution in both basal and luminal layers (arrow and arrowhead in d). Scale bar, 20 μm. Clonal analysis indicated that bi-lineage clones are the majority in all clones (97.7%) (e, f), and in two-cell clones (97.1%) (g). n=5 mice. c, Data are presented as mean±s.d. h-n, Tamoxifen was administered in P0.5 Procr^{CreERT2/+};R26^{mTmG/+} mice. Labelled cell contribution was analysed after 8-week induction (h). FACS analysis indicated that GFP⁺ cells are distributed in both basal and luminal layers (i, j). Immunostaining in sections showed the clonal expansion of GFP⁺ cells and confirmed their distribution in both basal and luminal layers. Basal cells were marked by K14, while cells apical to K14⁺ cells were luminal cells (arrows and arrowhead in k). Scale bar, 20 μm. Clonal analysis indicated that bi-lineage clones are the majority in all clones (98.5%) (l, m), and in two-cell clones (94.4%) (n). n=5 mice. j, Data are presented as mean±s.d.

FIGS. 13a-13k: Procr labels multipotent mammary stem cells in prepubescent mice.

a-d, Tamoxifen (TAM) was administered in 2-week old Procr^{CreERT2/+}; R26^{mTmG/+} mice. Labelled cell contribution was analysed at 8 weeks. FACS analysis indicated that GFP⁺ cells are distributed in both basal and luminal layers (b, c). Immunostaining in sections showed the clonal expansion of GFP⁺ cells and confirmed their distribution in both basal and luminal layers. Basal cells were marked by K14, while cells apical to K14⁺ cells were luminal cells (arrow and arrowhead in d). e-g, Clonal analysis indicated that bi-lineage clones are the majority in all clones (89.6%) (e, f), and in two-cell clones (78.8%) (g). n=4 mice. h-k, Mammary glands were analysed at day 14.5 gestation after tamoxifen administration at 2 weeks. GFP⁺ cells were in both basal and luminal layers as indicated by FACS analysis (i, j). GFP⁺ cells contributed to alveologenesis by immunohistochemistry analysis in sections (k). Scale bars, 20 μm. c, j, Data are presented as mean±s.d.

FIGS. 14a-14i: Procr⁺ cells are important for mammary development.

a, Schematic illustration of targeted ablation of Procr⁺ cells using the Procr-CreERT2 model to drive expression of DTA. b, Tamoxifen (TAM) was administered every 3 days a total of three times followed by analysing the 4th mammary gland. c-e, Whole-mount imaging of the mammary epithelium at P42.

The lymph node (L.N.) is indicated. Both the oil-treated control mammary epithelium (c) and the tamoxifen-treated R26^{DTA/1} control mammary epithelium (d) had grown to the distal edge of the fat pad. Tamoxifen administration in Procr^{CreERT2/1}: R26^{DTA/1} mice largely prevented the growth of the epithelium: the forefront of the epithelium halted at a position close to where the forefront was at the initiation of cell ablation (slightly past the lymph node) (e). Scale bars, 2 mm. f, Quantification of the distance from the epithelium forefront to the lymph node indicated that epithelium extension in the Procr⁻ cell-ablation group is largely compromised (comparing e with d or c). ***P<0.01. NS, not significant. g, h, FACS analysis indicating that Procr⁺ basal and stromal cells are ablated (fourfold and twofold). n=3 mice. The role of Procr⁺ stromal cells in this study should also be taken into consideration as they were also affected by the ablation. Nonetheless, the reduction of Procr⁺ stromal cells was not as pronounced as Procr⁺ basal cells, probably due to the less proliferative nature of Procr⁺ fibroblasts, thereby fewer progeny cells were affected. Data are presented as mean±s.d. ***P<0.01. i, Multipotent and unipotent MaSCs coexist in the mammary epithelial cell hierarchy. Multipotent MaSCs are characterized by Lin⁻CD24⁻CD29^{hi} Procr⁺K5^{low} K14^{low}, and express EMT features. Multipotent MaSCs generate all differentiated cell types, as determined by lineage tracing, and display the highest repopulation efficiency by transplantation. Basal-committed MaSCs are destined for basal cells in development, yet can repopulate both basal and luminal cells in transplantation, underlining the plasticity of basal-committed MaSCs in response to intervention. Luminal progenitors contribute to only luminal cells in lineage tracing and are not able to repopulate in transplantation. Their markers are as previously reported^{3, 4, 27}.

FIGS. 15a-15f. Procr is required for MaSCs and mammary development

(a) Isolated MaSCs were virally infected with control and two independent Procr-shRNA constructs followed by plating in matrigel culture. Colony sizes were measured at day 7 of culture. Knockdown of Procr inhibited MaSC colony formation. Scale bar represents 50 um. Data are presented as mean±s.e.m. Student's t test: ***p<0.0001.

(b) Schematic illustration of Procr deletion strategy in the Procr^{CreER/flox} model, using Procr-CreER in one knock-in allele to delete Procr exon 2-4 in the other allele.

(c) Whole-mount imaging of the Procr^{CreER/flox} mammary epithelium at 8-week old indicating a normal morphology. Scale bar represents 2 mm.

(d) 2-week old mice were administered with TAM for 3 doses (at d14, d16, d18), the 4^{th} mammary glands were analyzed at 8-week old as illustrated. The Procr^{CreER/flox} mammary gland displayed undeveloped branches, while the control Procr^{flox/+} mammary glands had normal epithelium extension and morphology. The lymph node (L.N.) is indicated. Scale bar represents 2 mm.

(e) qPCR analysis of the 3^{rd} mammary glands of mice in (d) indicating the successful deletion of Procr in Procr^{CreER/flox} mice.

(f) 8-week old mice were administered with TAM for 3 doses (at d56, d58, d60) and the 4^{th} mammary glands were analyzed at 11-week old. The Procr^{CreER/flox} mammary gland exhibited hollow and dilated ducts with reduced side branches, while the control Procr^{flox/+} mammary gland was normal. Scale bar represents 2 mm.

FIGS. 16a-16i. Procr is critical for mammary tumor formation

(a) qPCR analysis of Procr expression in four different mouse mammary tumors as indicated. Data are pooled from two independent experiments and presented as mean±s.e.m.

(b) FACS isolation of Procr+ and Procr− cells from basal cell population in MMTV-Wnt1 tumor. Procr+ basal cells consisted of 8.8±2% of total basal cells. Data are pooled from three independent experiments.

(c-e) Procr+ and Procr− basal cells (2,000 each) were engrafted to recipient fat pads. Procr+ formed tumor vigorously while Procr− cells could not. Representative pictures are shown in (c). Tumor volume and tumor free percentage are shown in (d) and (e) respectively. When the number of engrafted Procr− basal was increased to 10,000 cells, no tumor formation was observed (d, e). n=4 mice for each group.

(f) qPCR analysis indicating the Sh-Procr knock down efficacy in MMTV-Wnt1 mammary cells.

(g-i) MMTV-Wntl mammary cells was virally infected by scramble control or Sh-Procr followed by transplantation to the recipients. Control cells efficiently formed tumors, whereas knockdown of Procr inhibited tumor growth. Representative pictures are shown in (g). Tumor volume and tumor free percentage are shown in (h) and (i) respectively. n=4 mice for each group.

FIGS. 17a-17h. Procr is positively correlated with TNBC and is associated with poor clinical outcome

(a) Immunohistochemical staining of PROCR in the four subtypes of human breast cancer tissue samples. Representative of each subtype was shown. PROCR staining is in brown, hematoxylin counterstain is in blue. Scale bar represents 200 um.

(b) H-score analysis revealed a strong association of PROCR expression with TNBC. Student's t test: ***p<0.0001.

(c) PROCR expression was measure by Immunohistochemical staining in tissue microarray containing 71 no-cancerous, 100 luminal A cancers, 100 luminal B cancers, 100 Her2 cancers and 149 TNBCs. Representative of non-cancerrous, negative (score 0), weak (score 1), medium (score 2) and strong (score 3) staining are shown. Scale bars represent 200 μm in lower magnification, 50 um in the zoom in.

(d) Statistical analysis of PROCR expression according to the IHC score (d). More than 60% of TNBCs had medium and high PROCR expression, whereas majority of non-cancerous tissues and other subtypes are negative.

(e-f) Kaplan-Meier analysis of disease free survival (DFS) in our TNBC cohort. PROCR expression is associated with poor DFS in TNBC patients (e), but have no significant association without stratification of molecular subtypes (f).

(g-h) Kaplan-Meier analysis of DFS derived from a large public clinical database (kmplot.com). PROCR expression is associated with poor DFS in hormone-receptor negative breast cancer patients (g), not associated with hormone-receptor positive breast cancer patients (h).

FIGS. 18a-18l. Inhibition of PROCR suppresses TNBC formation

(a) qPCR analysis of PROCR expression in breast cancer cell lines as indicated. PROCR level is markedly higher in all three TNBC lines comparing to cells of other subtypes. Representative result is shown for three independent experiments.

(b) MDA-MB-231 cells were virally infected by scramble control or Sh-PROCR. An aliquot of cells were used to validate the PROCR knockdown by Western analysis.

(c) Immunostaining of MDA-MB-231 cells indicating that the spindle-shaped morphology is altered to spherical looking with PROCR knockdown. Cell shape was outlined by Vimentin staining (red) in the cytoplasm. Scale bar represents 20 um.

(d-e) MDA-MB-231 xenograft experiment indicating that knockdown of PROCR inhibits MDA-MB-231 tumor growth (d) and delay tumor formation (e). n=4 mice in each group. Data are presented as mean±s.e.m.

(f) IHC of PDX-3 tumor indicating its ER, PR, HER2 and PROCR status. Scale bar represents 100 um.

(g-i) PDX-3 tumor cells were virally infected by scramble control or Sh-PROCR. An aliquot of cells were used for Western analysis and confirmed 70% of PROCR knockdown (g). Xenografts of the infected cells indicating that PROCR knockdown blocks PDX-3 tumor growth (h, i). n=4 mice in each group. Data are presented as mean±s.e.m.

(j) Elisa indicating that the neutralizing antibody blocks PROCR and PROC binding, while control antibody cannot.

(k) The spindle-shape of MDA-MB-231 cells is altered to spherical morphology in the presence of the PROCR neutralizing antibody. Cell shape was outlined by cytoplasmic Vimentin staining (red). Scale bar represents 20 um.

(I) PDX-3 tumor cells were inoculated and antibodies were i.p. administered at d5, d7, d10, d14 and d19. Tumor sizes were suppressed with PROCR neutralizing antibody. n=3 mice in each group. Data are presented as mean±s.e.m.

FIGS. 19a-19b. Validation of mProcr and hPROCR ShRNAs by Western Blotting

(a) 293T cells were co-transfected with mouse Flag-Procr and Procr shRNA-1 or shRNA-2. Western analysis using Flag antibody indicating that both shRNAs efficiently inhibits Procr expression. Actin was used as a loading control. Sh-Procr represents Procr shRNA-1 unless specified otherwise.

(b) 293T cells were co-transfected with human Flag-PROCR and PROCR shRNA-1, shRNA-2 or shRNA-3. Western analysis using Flag antibody indicating that shRNA-1 completely knocked down PROCR expression, and shRNA-3 has partial knockdown efficacy. Actin was used as a loading control. Sh-PROCR represents PROCR shRNA-1 unless specified otherwise.

FIGS. 20a-20b. Generation of Procr conditional deletion allele

(a) Schematic illustration of targeting stratagy of Procr^{flox}. A loxP site was inserted upstream of exon 2, and an frt-flanked PGK-neo cassette followed by a second loxP site was inserted downstream of exon 4 of Procr gene. Loci of genotyping primers are shown.

(b) PCR genotyping results using 2 sets of primers indicating that pup #1, 2, 7, 8, 9 are Procr^{flox/-}, and that pup #3-6 are wildtype.

FIG. 21. Knockdown of PROCR inhibits MDA-MB-231 cell proliferation

MDA-MB-231 cells were virally infected with Scamble control and two individual PROCR shRNAs (shRNA-1 and shRNA-3) and culture for 4 passage in complete media. Cell numbers were counted in each passage. Both shRNAs could inhibit MDA-MB-231 cell proliferation.

FIGS. 22a-22c. PROCR extracellular domain and PROC kinase activity are important for MDA-MB-231 cell morphology

Schematic and immunostaining of MDA-MB-231 cells indicating that the spindle-shaped morphology in control, an indication of prone to migration/metastasis property (a) is altered to spherical looking in the presence of either sPROCR (b) or Protein C-kinase dead purified recombinant proteins (c). Cell shape was outlined by cytoplasmic Vimentin staining (red). Scale bar represents 20 um.

FIGS. 23a-23c. Knockdown of PROCR is ineffective in blocking MCF-7 tumor formation

(a) qPCR analysis indicating although MCF-7 exhibits lower PROCR expression compared to MDA-MB-231 cells, shRNA can further inhibit PROCR expression in MCF-7 cells.

(b-c) Xenograft experiments indicating that knockdown of PROCR does not affect MCF-7 tumor growth (b, c). n=4 mice in each group. Data are presented as mean±s.e.m.

FIGS. 24a-24c. Immunohistochemical staining of PDX samples

Immunohistochemical staining indicating that PDX-1 (a), PDX-2 (b) and PDX-3 (c) are all ER−, PR−, HER2−, and PROCR+.

FIGS. 25a-25f. Inhibition of PROCR suppresses TNBC PDX tumor formation

Tumor cells from PDX-1 (a-c) and PDX-2 (d-f) were virally infected by scramble control or Sh-PROCR. An aliquot of cells were use for Western analysis and confirmed the 50% of PROCR knockdown in PDX-1 (a) 70% of PROCR knockdown in PDX-2 (d). Xenografts of the infected cells indicating that PROCR knockdown blocks both PDX tumor growth (b, c, e, f). n=4 mice in each group. Data are presented as mean±s.e.m.

FIG. 26. Treatment with a PROCR neutralizing antibody inhibits PDX tumor growth

PDX-2 tumor cells were inoculated and control antibody or neutralizing antibody were i.p. administered at d5, d7, d10, d14 and d19. Tumor growth was inhibited with a PROCR neutralizing antibody. n=3 mice in each group. Data are presented as mean±s.e.m.

FIGS. 27a-27f. PROCR neutralizing antibody RCR-252

(a) Elisa assay indicating that a neutralizing antibody RCR-252 (ABCAM, ab81712) blocks the interaction of PROCR and PROC, whereas the control antibody (ABCAM, ab56689) cannot. (b) Western analysis indicating that RCR-252 detects overexpression of PROCR, whereas the control antibody cannot. (c) immunohistochemistry indicating that the control antibody detects PROCR (brown) in human TNBC tissue, while RCR-252 cannot. (d) RCR-252 inducing morphological change in TNBC (MDA-MB-231) cells, while the control antibody cannot. (e) Administration of RCR-252 into Patient Derived Xenograft models inhibited tumor growth, whereas the control antibody cannot.

### DETAILED DESCRIPTION

It is to be understood that both the foregoing general description and the following detailed description are exemplary and explanatory only and are not restrictive of the compositions and methods of the invention described herein.

One aspect of the present disclosure relates to the surprising discovery that Procr can be used as a biomarker and therapeutic target specifically for TNBC. PROCR is highly expressed in TNBC cells but not other subtypes of breast cancer. Furthermore, PROCR expression is highly correlated with (a) poor survival rate of breast cancer patients, (b) increased stemness of cancer stem cells and (c) metastasis in tumor models. It is additionally established herein that inhibition of PROCR defeats the tumorigenicity and progression of TNBC subtype. As such, PROCR can be used as an effective target for TNBC diagnosis and therapeutic intervention.

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

As used herein, the following terms and phrases are intended to have the following meanings:

The articles “a” and “an” are used herein to refer to one or to more than one (i.e., to at least one) of the grammatical object of the article. By way of example, “an element” means one element or more than one element.

As used herein, the term “about” means acceptable variations within 20%, more preferably within 10% and most preferably within 5% of the stated value.

As used herein, the term “triple negative” or “TN” or “TNBC” refers to tumors (e.g., carcinomas), typically breast tumors, in which the tumor cells score negative (i.e., using conventional histopathology methods) for estrogen receptor (ER) and progesterone receptor (PR), both of which are nuclear receptors (i.e., they are predominantly located at cell nuclei), and the tumor cells are not amplified for epidermal growth factor receptor type 2 (HER2 or ErbB2), a receptor normally located on the cell surface. Tumor cells are considered negative for expression of ER and PR if less than 5% of the tumor cell nuclei are stained for ER and PR expression using standard immunohistochemical techniques. Tumor cells are considered highly amplified for HER2 (“HER2³⁺”) if, when tested with a HercepTest™ Kit (Code K5204, Dako North America, Inc., Carpinteria, Calif.), a semi-quantitative immunohistochemical assay using a polyclonal anti-HER2 primary antibody, they yield a test result score of 3+, or, the test HER2 positive by fluorescence in-situ hybridization (FISH). As used herein, tumor cells are considered negative for HER2 overexpression if they yield a test result score of 0 or 1+, or 2+, or if they are HER2 FISH negative.

“Epithelial-mesenchymal transition” or “EMT” refers to the loss of epithelial characteristics and the acquisition of a mesenchymal phenotype. In this process, cells acquire molecular alterations that facilitate dysfunctional cell-cell adhesive interactions and junctions. These processes may promote cancer cell progression and invasion into the surrounding microenvironment. Such transformation has implications in progression of breast carcinoma to metastasis, and increasing evidences support most tumors contain a subpopulation of cells with stem-like and mesenchymal features that is resistant to chemotherapy.

“Metastasis” refers to the process by which cancer spreads from the place at which it first arose as a primary tumor (e.g., breast cancer) to distant locations in the body. Metastasis depends on the cancer cells acquiring two separate abilities—increased motility and invasiveness.

“Procr” and “PROCR” are used interchangeably and refer to protein C receptor, with “Procr” generally referring to the gene and “PROCR” the protein product unless otherwise noted. It should be understood that the terms include the complete gene, the cDNA sequence, the complete amino acid sequence, or any fragment or variant thereof.

As used herein, the term “PROCR inhibitor” is intended to include therapeutic agents that inhibit, down-modulate, suppress or down-regulate PROCR activity. The term is intended to include chemical compounds, such as small molecule inhibitors and biologic agents (e.g., antibodies), interfering RNA (shRNA, siRNA), soluble antagonists, gene editing/silencing tools (CRISPR/Cas9, TALENs) and the like.

An “antibody,” as used herein is a protein consisting of one or more polypeptides comprising binding domains substantially encoded by immunoglobulin genes or fragments of immunoglobulin genes, wherein the protein immunospecifically binds to an antigen. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin structural unit comprises a tetramer that is composed of two identical pairs of polypeptide chains, each pair having one “light” (about 25 kD) and one “heavy” chain (about 50-70 kD). “V_{L}” and V_{H}″ refer to these light and heavy chains respectively.

Antibodies include intact immunoglobulins as well as antigen-binding fragments thereof, which may be produced by digestion with various peptidases, or synthesized de novo either chemically or using recombinant DNA expression technology. Such fragments include, for example, F(ab)₂ dimers and Fab monomers. Useful antibodies include single chain antibodies (antibodies that exist as a single polypeptide chain), e.g., single chain Fv antibodies (scFv) in which a V_{H} and a V_{L} chain are joined together (directly or through a peptide linker) to form a continuous polypeptide.

Antibodies also include variants, chimeric antibodies and humanized antibodies. The term “antibody variant” as used herein refers to an antibody with single or multiple mutations in the heavy chains and/or light chains. In some embodiments, the mutations exist in the variable region. In some embodiments, the mutations exist in the constant region. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non-human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example. “Humanized” antibodies refer to a molecule having an antigen-binding site that is substantially derived from an immunoglobulin from a non-human species and the remaining immunoglobulin structure of the molecule based upon the structure and/or sequence of a human immunoglobulin. The antigen-binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, or six) which are altered with respect to the original antibody, which are also termed one or more CDRs “derived from” one or more CDRs.

An “anti-PROCR antibody” is an antibody that immunospecifically binds to PROCR (e.g., its extracellular domain). The antibody may be an isolated antibody. Such binding to PROCR exhibits a K_{d} with a value of, e.g., no greater than 1 μM, no greater than 100 nM or no greater than 50 nM. Kd can be measured by any methods known to a skilled in the art, such as a surface plasmon resonance assay or a cell binding assay. An anti-PROCR antibody may be an isolated antibody. Exemplary anti-PROCR antibodies inhibit PROCR binding with protein C.

“RCR-252” refers to the monoclonal antibody having clone number RCR-252 as first described in Ye et al, “The endothelial cell protein C receptor (EPCR) functions as a primary receptor for protein C activation on endothelial cells in arteries, veins, and capillaries,” Biochem Biophys Res Commun 1999, 259: 671. RCR-252 is a rat anti human PROCR antibody, and is commercially available from multiple sources, such as Abcam under Catalog No. ab81712 and Sigma under Product No. E6280.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

“Immunospecific” or “immunospecifically” refer to antibodies that bind via domains substantially encoded by immunoglobulin genes or fragments of immunoglobulin genes to one or more epitopes of a protein of interest, but which do not substantially recognize and bind other molecules in a sample containing a mixed population of antigenic molecules. Typically, an antibody binds immunospecifically to a cognate antigen with a K_{d} with a value of no greater than 50 nM, as measured by a surface plasmon resonance assay or a cell binding assay. The use of such assays is well known in the art.

The terms “cross-compete”, “cross-competition”, “cross-block”, “cross-blocked” and “cross-blocking” are used interchangeably herein to mean the ability of an antibody or fragment thereof to interfere with the binding directly or indirectly through allosteric modulation of the anti-PROCR antibodies of the present disclosure to the target PROCR. The extent to which an antibody or fragment thereof is able to interfere with the binding of another to the target, and therefore whether it can be said to cross-block or cross-compete according to the present disclosure, can be determined using competition binding assays. One particularly suitable quantitative cross-competition assay uses a FACS− or an AlphaScreen-based approach to measure competition between the labelled (e.g. His tagged, biotinylated or radioactive labelled) an antibody or fragment thereof and the other an antibody or fragment thereof in terms of their binding to the target. In general, a cross-competing antibody or fragment thereof is for example one which will bind to the target in the cross-competition assay such that, during the assay and in the presence of a second antibody or fragment thereof, the recorded displacement of the immunoglobulin single variable domain or polypeptide according to the invention is up to 100% (e.g., in FACS based competition assay) of the maximum theoretical displacement (e.g., displacement by cold (e.g., unlabeled) antibody or fragment thereof that needs to be cross-blocked) by the to be tested potentially cross-blocking antibody or fragment thereof that is present in a given amount. Preferably, cross-competing antibodies or fragments thereof have a recorded displacement that is between 10% and 100%, more preferred between 50% to 100%.

The terms “suppress”, “suppression”, “inhibit”, “inhibition”, “neutralize” and “neutralizing” as used interchangeably herein, refer to any statistically significant decrease in biological activity (e.g., PROCR activity or tumor cell growth), including full blocking of the activity. For example, “inhibition” can refer to a decrease of about 10%, 20%, 30%, 40%, 50%, 60%, 70%, 80%, 90%, or 100% in biological activity.

The term “patient” includes a human or other mammalian animal that receives either prophylactic or therapeutic treatment.

The terms “treat,” “treating,” and “treatment,” as used herein, refer to therapeutic or preventative measures such as those described herein. The methods of “treatment” employ administration to a patient of a PROCR inhibitor provided herein, for example, a patient having TNBC, in order to prevent, cure, delay, reduce the severity of, or ameliorate one or more symptoms of the disease or disorder or recurring disease or disorder, or in order to prolong the survival of a patient beyond that expected in the absence of such treatment.

The term “effective amount,” as used herein, refers to that amount of an agent, such as a PROCR inhibitor, for example an anti-PROCR antibody, which is sufficient to effect treatment, prognosis or diagnosis of TNBC, when administered to a patient. A therapeutically effective amount will vary depending upon the patient and disease condition being treated, the weight and age of the patient, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1μg to about 3,500 mg, about 5μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of an antibody or antigen binding portion thereof, as provided herein. Dosing may be, e.g., every week, every 2 weeks, every three weeks, every 4 weeks, every 5 weeks or every 6 weeks. Dosage regimens may be adjusted to provide the optimum therapeutic response. An effective amount is also one in which any toxic or detrimental effects (side effects) of the agent are minimized and/or outweighed by the beneficial effects. Administration may be intravenous at exactly or about 6 mg/kg or 12 mg/kg weekly, or 12 mg/kg or 24 mg/kg biweekly. Additional dosing regimens are described below.

Other terms used in the fields of recombinant nucleic acid technology, microbiology, immunology, antibody engineering, and molecular and cell biology as used herein will be generally understood by one of ordinary skill in the applicable arts. For example, conventional techniques may be used for preparing recombinant DNA, performing oligonucleotide synthesis, and practicing tissue culture and transformation (e.g., electroporation, transfection or lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., 2001, Molecular Cloning: A Laboratory Manual, 3rd ed., Cold Spring Harbor Laboratory Press, Cold Spring Harbor, N.Y., which is incorporated herein by reference for any purpose. Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Various aspects and embodiments are described in further detail in the following subsections.

PROCR and Use as TNBC Biomarker

The human PROCR is a highly glycosylated type I transmembrane protein of 238 amino-acids (UniProtKB ID No. Q9UNN8; SEQ ID NO.: 2). These amino acids comprise a signal peptide (amino acids 1017), an extracellular domain (amino acids 18-210), a 21-aa transmembrane domain (amino acids 211-231), and a 7-aa intracytoplasmic sequence (amino acids 232-238) together coding for an ˜46 kDa protein. Deglycosylation will reduce the protein mass to 25 kDa. PROCR is expressed strongly on the endothelial cells of arteries and veins in heart and lung, less intensely in capillaries in the lung and skin, and not at all in the endothelium of small vessels of the liver and kidney.

PROCR is the receptor for protein C, a key player in the anticoagulation pathway. The protein C anticoagulant pathway serves as a major system for controlling thrombosis, limiting inflammatory responses, and potentially decreasing endothelial cell apoptosis in response to inflammatory cytokines and ischemia. The essential components of the pathway include thrombin, thrombomodulin, PROCR, protein C and protein S. The pathway is initiated when thrombin binds to thrombomodulin on the surface of endothelium. PROCR augments protein C activation by binding protein C and presenting it to the thrombin-thrombomodulin activation complex. Activated protein C (aPC) retains its ability to bind PROCR, and this complex appears to be involved in some of the cellular signaling mechanisms that down-regulate inflammatory cytokine formation (TNF, IL-6). PROCR is shed from the vasculature by inflammatory mediators and thrombin. PROCR binds to activated neutrophils in a process that involves proteinase 3 and Mac-1. Furthermore, PROCR can undergo translocation from the plasma membrane to the nucleus.

PROCR can be cleaved to release a soluble form (sPROCR) in the circulation. This sPROCR is detected as a single species of 43 kDa, resulting from shedding of membrane PROCR by the action of a metalloprotease, which is stimulated by thrombin and by some inflammatory mediators. Soluble PROCR binds PC and aPC with similar affinity, but its binding to aPC inhibits the anticoagulant activity of aPC by blocking its binding to phospholipids and by abrogating its ability to inactivate factor Va. sPROCR can be detected in plasma. In normal persons, sPROCR is present in levels of 83.6+/−17.2 ng/ml. Elevated levels of sPROCR are positively correlated to a higher risk for thrombosis. Furthermore, a haplotype (A3 allele) has been linked to elevated levels of sPROCR (264 +/−174 ng/ml).

The full gene sequence of human Procr is 44819 bp (GenBank ID No. NC_000020.11). The human cDNA sequence is 717 bp in length (GenBank ID No. NM_006404.4; SEQ ID NO.:1). The full gene sequence of mouse Procr gene is 4354 bp (GenBank ID No. NC_000068.7). The mouse cDNA sequence is 729 bp in length (GenBank ID No.NM_0111171.2; SEQ ID NO.:3). The mouse PROCR protein sequence has UniProtKB ID No. Q64695 (SEQ ID NO.: 4).

In some embodiments, the presence of PROCR and/or its expression level can be used as a biomarker for diagnosing and/or determining the prognosis of TNBC. This is based on the surprising discovery that PROCR level or Procr gene expression level is elevated in TNBC cells but not other subtypes of breast cancer.

PROCR protein level can be measured by mass spectrometry or an immunoassay using an anti-PROCR antibody, such as immunohistochemistry on a tissue sample or enzyme linked immunosorbent assay (ELISA) or Western blot. Alternatively, PROCR mRNA level can be measured by quantitative reverse transcription PCR (qRT-PCR) or Northern blot or microarray. Other methods known in the art can also be used to detect the presence of PROCR and/or measure its expression level.

Kits for detecting PROCR and thus, diagnosing TNBC are also provided. The kit can include one or more anti-PROCR antibody disclosed herein, or antigen binding fragment thereof, for use in connection with an immunoassay such as immunohistochemistry or ELISA or Western blot. Alternatively, the kit can include specific primers and/or probes for use in connection with qRT-PCR (e.g., using primers of 10-30 bp designed to target SEQ ID NO.:1) or Northern blot (e.g., using probes of 30-300 bp designed to target SEQ ID NO.:1). The kit can also include a microarray for detecting Procr mRNA or protein level where Procr gene or a fragment thereof, or anti-PROCR antibody or an antigen binding fragment thereof, can be attached to the microarray. A control sample along with a user instruction manual can additionally be included in the kit, wherein a difference (e.g., increase) in the test sample compared to the control sample (after normalization) indicates the presence of TNBC. The increase can be more than about 10%, more than about 20%, more than about 30%, more than about 50%, more than about 60%, more than about 80%, more than about 100%, or more, or any number therebetween.

PROCR Inhibitor and Use Thereof

In addition to being a strong marker for TNBC, the functional relevance of PROCR expression in TNBC is also unexpected. PROCR expression is highly correlated with (a) poor survival rate of breast cancer patients, (b) increased stemness of cancer stem cells and (c) metastasis in tumor models. These observations suggest PROCR plays an important role in tumorigenicity and progression of TNBC. Indeed, inhibition of PROCR defeats the tumorigenicity and progression of TNBC subtype. As such, PROCR inhibitors can be used as effective TNBC therapeutics.

Various PROCR inhibitors are included in the present disclosure. Examples include chemical compounds, such as small molecule inhibitors and biologic agents (e.g., antibodies) that can bind PROCR and inhibit or decrease its activity, e.g., binding to protein C. Agents that regulate Procr gene expression level are also included, such as interfering RNA (shRNA, siRNA) and gene editing/silencing tools (CRISPR/Cas9, TALENs, zinc finger nucleases) that are designed specifically to target the Procr gene or a regulatory sequence thereto.

In certain embodiments, the PROCR inhibitor is an anti-PROCR antibody, e.g., a monoclonal antibody. An exemplary anti-PROCR antibody is RCR-252, described in Ye et al; The endothelial cell protein C receptor (EPCR) functions as a primary receptor for protein C activation on endothelial cells in arteries, veins, and capillaries. Biochem Biophys Res Commun 1999, 259: 671. Alternately, the anti-PROCR antibody can be an antibody that cross-competes with RCR-252 for binding to PROCR. In another embodiment, the anti-PROCR antibody is an antibody comprising the V_{H} and V_{L} CDR sequences of RCR-252.

In some embodiments, the anti-PROCR antibody is an antibody or antigen binding portion thereof which binds an epitope of human PROCR, e.g., the extracellular domain. The epitope may be bound by RCR-252, or RCR-252 binds to a different but proximate epitope on PROCR. The anti-PROCR antibody can be characterized by at least partial inhibition of proliferation (e.g., by at least 10% relative to control) of a cancer cell expressing PROCR or by at least partial inhibition of tumor growth (e.g., volume and/or metastasis) in vivo in the patient or in a patient-derived xenograft.

In yet another embodiment, the anti-PROCR antibody can comprise a mixture, or cocktail, of two or more anti-PROCR antibodies, each of which binds to a different epitope on PROCR. In one embodiment, the mixture, or cocktail, comprises three anti-PROCR antibodies, each of which binds to a different epitope on PROCR.

In another embodiment, the PROCR inhibitor comprises a nucleic acid molecule, such as an RNA molecule, that inhibits the expression or activity of PROCR. Interfering RNAs specific for Procr, such as shRNAs or siRNAs that specifically inhibits the expression and/or activity of Procr, can be designed in accordance with methods known in the art.

In some embodiments, PROCR-expressing cells (e.g., TNBC cells) or a patient-derived xenograft can be used as a model for screening for agents that inhibit PROCR expression and/or activity. An exemplary method includes: (a) providing a test agent to a plurality of PROCR-expressing TNBC cells, and (b) determining one or more of (1) PROCR expression level, (2) PROCR activity, and (3) survival and/or proliferation rate of the TNBC cells, wherein a decrease compared to a negative control not treated by the test agent indicates that the test agent is a PROCR inhibitor. Another exemplary method includes: (a) providing a test agent to a patient-derived xenograft, and (b) determining (1) PROCR expression level, (2) PROCR activity, and (3) tumor growth and/or metastasis in the xenograft, wherein a decrease compared to a negative control not treated by the test agent indicates that the test agent is a PROCR inhibitor. Yet another exemplary method includes: (a) providing a test agent, and (b) determining whether the test agent has one or more of the following characteristics: (i) binding to PROCR; (ii) interfering with or inhibiting binding of PROCR with protein C; (iii) cross-competing with RCR-252; (iv) interfering with or inhibiting binding of RCR-252 with PROCR; and/or (v) enhancing binding of RCR-252 with PROCR; wherein the test agent is a PROCR inhibitor if it has one or more of (i)-(v). These methods can be performed in vitro or in vivo. The test agent can be an antibody, a small molecule, a peptide and/or a nucleic acid.

In one aspect, use of a PROCR inhibitor for the manufacture of a medicament for the treatment of TNBC is provided. In another aspect, a method of suppressing growth of a TNBC cell is provided, the method comprising contacting the cell with an effective amount of a PROCR inhibitor. In another aspect, a method of suppressing growth of a TNBC tumor in a patient is provided, the method comprising administering to the patient an effective amount of a PROCR inhibitor. In yet another aspect, a method of treating a patient for a TNBC tumor is provided, the method comprising administering to the patient an effective amount of a PROCR inhibitor. In still another aspect, a method of treating a breast cancer tumor in a patient is provided, the method comprising: selecting a patient with a TNBC tumor; and administering to the patient an effective amount of a PROCR inhibitor. In one embodiment of any of the above methods, the PROCR inhibitor is an anti-PROCR antibody. An exemplary anti-PROCR antibody is RCR-252 or an antigen binding fragment thereof, or an antibody that cross-competes with RCR-252 in PROCR binding

Preparation of Anti-PROCR Antibodies

Antibodies typically comprise two identical pairs of polypeptide chains, each pair having one full-length “light” chain (typically having a molecular weight of about 25 kDa) and one full-length “heavy” chain (typically having a molecular weight of about 50-70 kDa). The amino-terminal portion of each chain typically includes a variable region of about 100 to 110 or more amino acids that typically is responsible for antigen recognition. The carboxy-terminal portion of each chain typically defines a constant region responsible for effector function. The variable regions of each of the heavy chains and light chains typically exhibit the same general structure comprising four relatively conserved framework regions (FR) joined by three hyper variable regions, also called complementarity determining regions or CDRs. The CDRs from the two chains of each pair typically are aligned by the framework regions, which alignment may enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (1987 and 1991, National Institutes of Health, Bethesda, Md.), Chothia & Lesk, 1987, J. Mol. Biol. 196:901-917, or Chothia et al., 1989, Nature 342:878-883).

Antibodies became useful and of interest as pharmaceutical agents with the development of monoclonal antibodies. Monoclonal antibodies are produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, Marcel Dekker, Inc., New York, pp. 51-63).

Monoclonal antibodies may be modified for use as therapeutics. One example is a “chimeric” antibody in which a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Other examples are fragments of such antibodies, so long as they exhibit the desired biological activity. See, U.S. Pat. No. 4,816,567; and Morrison et al. (1985), Proc. Natl. Acad. Sci. USA 81:6851-6855. A related development is the “CDR-grafted” antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass.

Another development is the “humanized” antibody. Methods for humanizing non-human antibodies are well known in the art. (See U.S. Pat. Nos. 5,585,089, and 5,693,762). Generally, a humanized antibody is produced by a non-human animal, and then certain amino acid residues, typically from non-antigen recognizing portions of the antibody, are modified to be homologous to said residues in a human antibody of corresponding isotype. Humanization can be performed, for example, using methods described in the art (Jones et al., 1986, Nature 321:522-525; Riechmann et al., 1988, Nature 332:323-327; Verhoeyen et al., 1988, Science 239:1534-1536), by substituting at least a portion of a rodent variable region for the corresponding regions of a human antibody.

More recent is the development of human antibodies without exposure of antigen to human beings (“fully human antibodies”). Using transgenic animals (e.g., mice) that are capable of producing a repertoire of human antibodies in the absence of endogenous mouse immunoglobulin production, such antibodies are produced by immunization with an antigen (typically having at least 6 contiguous amino acids), optionally conjugated to a carrier. See, for example, Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of these methods, transgenic animals are produced by incapacitating the endogenous mouse immunoglobulin loci encoding the mouse heavy and light immunoglobulin chains therein, and inserting loci encoding human heavy and light chain proteins into the genome thereof. Partially modified animals, which have less than the full complement of modifications, are then cross-bred to obtain an animal having all of the desired immune system modifications. When administered an immunogen, these transgenic animals produce antibodies that are immunospecific for these antigens having human (rather than murine) amino acid sequences, including variable regions. See PCT Publication Nos. WO96/33735 and WO94/02602, incorporated by reference. Additional methods are described in U.S. Pat. No. 5,545,807, PCT Publication Nos. WO91/10741, WO90/04036, and in EP 546073B1 and EP 546073A1, incorporated by reference. Human antibodies may also be produced by the expression of recombinant DNA in host cells or by expression in hybridoma cells as described herein.

Fully human antibodies can also be produced from phage-display libraries (as disclosed in Hoogenboom et al., 1991, J. Mol. Biol. 227:381; and Marks et al., 1991, J. Mol. Biol. 222:581). These processes mimic immune selection through the display of antibody repertoires on the surface of filamentous bacteriophage, and subsequent selection of phage by their binding to an antigen of choice. One such technique is described in PCT Publication No. WO99/10494, incorporated by reference, which describes the isolation of high affinity and functional agonistic antibodies for MPL− and msk-receptors using such an approach.

Once the nucleotide sequences encoding the above antibodies have been determined, chimeric, CDR-grafted, humanized, and fully human antibodies also may be produced by recombinant methods. Nucleic acids encoding the antibodies are introduced into host cells and expressed using materials and procedures generally known in the art.

The invention provides one or a plurality of monoclonal antibodies against PROCR. Preferably, the antibodies bind PROCR. In preferred embodiments, the invention provides nucleotide sequences encoding, and amino acid sequences comprising, heavy and light chain immunoglobulin molecules, particularly sequences corresponding to the variable regions thereof. In preferred embodiments, sequences corresponding to CDRs, specifically from CDR1 through CDR3, are provided. In additional embodiments, the invention provides hybridoma cell lines expressing such immunoglobulin molecules and monoclonal antibodies produced therefrom, preferably purified human monoclonal antibodies against human PROCR.

The CDRs of the light and heavy chain variable regions of anti-PROCR antibodies of the invention can be grafted to framework regions (FRs) from the same, or another, species. In certain embodiments, the CDRs of the light and heavy chain variable regions of anti-PROCR antibody may be grafted to consensus human FRs. To create consensus human FRs, FRs from several human heavy chain or light chain amino acid sequences are aligned to identify a consensus amino acid sequence. The FRs of the anti-PROCR antibody heavy chain or light chain can be replaced with the FRs from a different heavy chain or light chain. Rare amino acids in the FRs of the heavy and light chains of anti-PROCR antibody typically are not replaced, while the rest of the FR amino acids can be replaced. Rare amino acids are specific amino acids that are in positions in which they are not usually found in FRs. The grafted variable regions from anti-PROCR antibodies of the invention can be used with a constant region that is different from the constant region of anti-PROCR antibody. Alternatively, the grafted variable regions are part of a single chain Fv antibody. CDR grafting is described, e.g., in U.S. Pat. Nos. 6,180,370, 5,693,762, 5,693,761, 5,585,089, and 5,530,101, which are hereby incorporated by reference for any purpose.

In certain embodiments, the invention provides an anti-PROCR antibody RCR-252. In other embodiments, the invention provides anti-PROCR antibodies that comprise a human light chain CDR1 region, a human heavy chain CDR2 region, and/or a human heavy chain CDR3 region of RCR-252.

In some embodiments, antibodies of the invention can be produced by hybridoma lines. In these embodiments, the antibodies of the invention bind to PROCR with a dissociation constant (K_{d}) of between approximately 4 pM and 1 μM. In certain embodiments of the invention, the antibodies bind to PROCR with a K_{d} of less than about 100 nM, less than about 50 nM or less than about 10 nM.

In preferred embodiments, the antibodies of the invention are of the IgG1, IgG2, or IgG4 isotype, with the IgG1 isotype most preferred. In preferred embodiments of the invention, the antibodies comprise a human kappa light chain and a human IgG1, IgG2, or IgG4 heavy chain. In particular embodiments, the variable regions of the antibodies are ligated to a constant region other than the constant region for the IgG1, IgG2, or IgG4 isotype. In certain embodiments, the antibodies of the invention have been cloned for expression in mammalian cells.

In alternative embodiments, antibodies of the invention can be expressed in cell lines other than hybridoma cell lines. In these embodiments, sequences encoding particular antibodies can be used for transformation of a suitable mammalian host cell. According to these embodiments, transformation can be achieved using any known method for introducing polynucleotides into a host cell, including, for example packaging the polynucleotide in a virus (or into a viral vector) and transducing a host cell with the virus (or vector) or by transfection procedures known in the art. Such procedures are exemplified by U.S. Pat. Nos. 4,399,216, 4,912,040, 4,740,461, and 4,959,455 (all of which are hereby incorporated herein by reference for any purpose). Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene-mediated transfection, protoplast fusion, electroporation, encapsulation of the polynucleotide(s) in liposomes, and direct microinjection of the DNA into nuclei.

According to certain embodiments of the methods of the invention, a nucleic acid molecule encoding the amino acid sequence of a heavy chain constant region, a heavy chain variable region, a light chain constant region, or a light chain variable region of a PROCR antibody of the invention is inserted into an appropriate expression vector using standard ligation techniques. In a preferred embodiment, the PROCR heavy or light chain constant region is appended to the C-terminus of the appropriate variable region and is ligated into an expression vector. The vector is typically selected to be functional in the particular host cell employed (i.e., the vector is compatible with the host cell machinery such that amplification of the gene and/or expression of the gene can occur). For a review of expression vectors, see, Goeddel (ed.), 1990, Meth. Enzymol. Vol. 185, Academic Press. N.Y.

Typically, expression vectors used in any of the host cells will contain sequences for plasmid maintenance and for cloning and expression of exogenous nucleotide sequences. Such sequences typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. These sequences are well known in the art.

Expression vectors of the invention may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding light chain or heavy chain or light chain and heavy chain comprising an anti-PROCR antibody has been inserted into the proper site of the vector, the completed vector may be inserted into a suitable host cell for amplification and/or polypeptide expression. The transformation of an expression vector for an anti-PROCR antibody into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., supra.

The host cell, when cultured under appropriate conditions, synthesizes an anti-PROCR antibody that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, one may select cell lines by determining which cell lines have high expression levels and produce antibodies with constitutive PROCR binding properties. In another embodiment, one may select a cell line from the B cell lineage that does not make its own antibody but has a capacity to make and secrete a heterologous antibody (e.g., mouse myeloma cell lines NS0 and SP2/0).

Pharmaceutical Compositions and Use Thereof

In another aspect, pharmaceutical compositions are provided that can be used in the methods disclosed herein, i.e., pharmaceutical compositions for treating TNBC.

In one embodiment, the pharmaceutical composition for treating TNBC comprises a PROCR inhibitor and a pharmaceutical carrier. The PROCR inhibitor can be formulated with the pharmaceutical carrier into a pharmaceutical composition. Additionally, the pharmaceutical composition can include, for example, instructions for use of the composition for the treatment of patients for TNBC.

In one embodiment, the PROCR inhibitor in the composition is an anti-PROCR antibody, e.g., RCR-252 or an antibody comprising the V_{H} and V_{L} CDRs of RCR-252 positioned in the antibody in the same relative order as they are present in RCR-252 so as to provide immunospecific binding of PROCR. In some embodiments, antibodies or antigen binding fragments thereof that can cross-compete with RCR-252 in PROCR binding are provided by the present disclosure.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it will be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration.

Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutical compositions comprising a PROCR inhibitor can be administered alone or in combination therapy. For example, the combination therapy can include a composition provided herein comprising a PROCR inhibitor and at least one or more additional therapeutic agents, such as one or more chemotherapeutic agents known in the art, discussed in further detail below. Pharmaceutical compositions can also be administered in conjunction with radiation therapy and/or surgery.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

Exemplary dosage ranges for administration of an antibody include: 10-1000 mg (antibody)/kg (body weight of the patient), 10-800 mg/kg, 10-600 mg/kg, 10-400 mg/kg, 10-200 mg/kg, 30-1000 mg/kg, 30-800 mg/kg, 30-600 mg/kg, 30-400 mg/kg, 30-200 mg/kg, 50-1000 mg/kg, 50-800 mg/kg, 50-600 mg/kg, 50-400 mg/kg, 50-200 mg/kg, 100-1000 mg/kg, 100-900 mg/kg, 100-800 mg/kg, 100-700 mg/kg, 100-600 mg/kg, 100-500 mg/kg, 100-400 mg/kg, 100-300 mg/kg and 100-200 mg/kg. Exemplary dosage schedules include once every three days, once every five days, once every seven days (i.e., once a week), once every 10 days, once every 14 days (i.e., once every two weeks), once every 21 days (i.e., once every three weeks), once every 28 days (i.e., once every four weeks) and once a month.

It may be advantageous to formulate parenteral compositions in unit dosage form for ease of administration and uniformity of dosage. Unit dosage form as used herein refers to physically discrete units suited as unitary dosages for the patients to be treated; each unit contains a predetermined quantity of active agent calculated to produce the desired therapeutic effect in association with any required pharmaceutical carrier. The specification for unit dosage forms are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Intravenous injection and infusion are often (but not exclusively) used for antibody administration.

When agents provided herein are administered as pharmaceuticals, to humans or animals, they can be given alone or as a pharmaceutical composition containing, for example, 0.001 to 90% (e.g., 0.005 to 70%, e.g., 0.01 to 30%) of active ingredient in combination with a pharmaceutically acceptable carrier.

In certain embodiments, the methods and uses provided herein for suppressing growth of TNBC cells or for treating a patient with TNBC can comprise administration of a PROCR inhibitor and at least one additional anti-cancer agent that is not a PROCR inhibitor.

In one embodiment, the at least one additional anti-cancer agent comprises at least one chemotherapeutic drug. Non-limiting examples of such chemotherapeutic drugs include platinum-based chemotherapy drugs (e.g., cisplatin, carboplatin), taxanes (e.g., paclitaxel (Taxol®), docetaxel (Taxotere®), EndoTAG-1™ (a formulation of paclitaxel encapsulated in positively charged lipid-based complexes; MediGene), Abraxane® (a formulation of paclitaxel bound to albumin)), tyrosine kinase inhibitors (e.g., imatinib/Gleevec®, sunitinib/Sutent®, dasatinib/Sprycel®), and combinations thereof

In another embodiment, the at least one additional anti-cancer agent comprises an EGFR inhibitor, such as an anti-EGFR antibody or a small molecule inhibitor of EGFR signaling. An exemplary anti-EGFR antibody is cetuximab (Erbitux®). Cetuximab is commercially available from ImClone Systems Incorporated. Other examples of anti-EGFR antibodies include matuzumab (EMD72000), panitumumab (Vectibix®; Amgen); nimotuzumab (TheraCIM™) and mAb 806. An exemplary small molecule inhibitor of the EGFR signaling pathway is gefitinib (Iressa®), which is commercially available from AstraZeneca and Teva. Other examples of small molecule inhibitors of the EGFR signaling pathway include erlotinib HCL (OSI-774; Tarceva®, OSI Pharma); lapatinib (Tykerb®, GlaxoSmithKline); canertinib (canertinib dihydrochloride, Pfizer); pelitinib (Pfizer); PKI-166 (Novartis); PD158780; and AG 1478 (4-(3-Chloroanillino)-6,7-dimethoxyquinazoline).

In yet another embodiment, the at least one additional anti-cancer agent comprises a VEGF inhibitor. An exemplary VEGF inhibitor comprises an anti-VEGF antibody, such as bevacizumab (Avastatin®; Genentech).

In still another embodiment, the at least one additional anti-cancer agent comprises an anti-ErbB2 antibody. Suitable anti-ErbB2 antibodies include trastuzumab and pertuzumab.

In one aspect, the improved effectiveness of a combination according to the invention can be demonstrated by achieving therapeutic synergy.

The term “therapeutic synergy” is used when the combination of two products at given doses is more efficacious than the best of each of the two products alone at the same doses. In one example, therapeutic synergy can be evaluated by comparing a combination to the best single agent using estimates obtained from a two-way analysis of variance with repeated measurements (e.g., time factor) on parameter tumor volume.

The term “additive” refers to when the combination of two or more products at given doses is equally efficacious than the sum of the efficacies obtained with of each of the two or more products, whilst the term “superadditive” refers to when the combination is more efficacious than the sum of the efficacies obtained with of each of the two or more products.

Another measure by which effectiveness (including effectiveness of combinations) can be quantified is by calculating the logᵢₒ cell kill, which is determined according to the following equation: log₁₀ cell kill=T−C (days)/3.32×T_{d} in which T−C represents the delay in growth of the cells, which is the average time, in days, for the tumors of the treated group (T) and the tumors of the control group (C) to have reached a predetermined value (1 g, or 10 mL, for example), and T_{d} represents the time, in days necessary for the volume of the tumor to double in the control animals. When applying this measure, a product is considered to be active if log₁₀ cell kill is greater than or equal to 0.7 and a product is considered to be very active if log₁₀ cell kill is greater than 2.8.

Using this measure, a combination, used at its own maximum tolerated dose, in which each of the constituents is present at a dose generally less than or equal to its maximum tolerated dose, exhibits therapeutic synergy when the logᵢₒ cell kill is greater than the value of the log₁₀ cell kill of the best constituent when it is administered alone. In an exemplary case, the logᵢₒ cell kill of the combination exceeds the value of the _{logio} cell kill of the best constituent of the combination by at least one log cell kill.

The following examples, including the experiments conducted and results achieved are provided for illustrative purposes only and are not to be construed as limiting the invention.

### EXAMPLE 1

### Identification of Multipotent Mammary Stem Cells by Protein C Receptor Expression

The mammary gland is composed of multiple types of epithelial cells, which are generated by mammary stem cells (MaSCs) residing at the top of the hierarchy^{1,2}. However, the existence of these multipotent MaSCs remains controversial and the nature of such cells is unknown^{3,4}. Here we demonstrate that protein C receptor (Procr), a novel Wnt target in the mammary gland, marks a unique population of multipotent mouse MaSCs. Procr-positive cells localize to the basal layer, exhibit epithelial-to-mesenchymal transition characteristics, and express low levels of basal keratins. Procr-expressing cells have a high regenerative capacity in transplantation assays and differentiate into all lineages of the mammary epithelium by lineage tracing. These results define a novel multipotent mammary stem cell population that could be important in the initiation of breast cancer.

The mammary gland is an epithelial organ consisting of myoepithelial (basal) cells and luminal cells. During pregnancy, the luminal cells at side branches undergo terminal differentiation and form alveolar cells. Previous studies using the surface markers Lin⁻, CD24⁺ and CD29^{hi} , and transplantation assays, indicate that MaSCs reside in the basal layer of the epithelium^{1,2}. This population is heterogeneous, including MaSCs, differentiated basal cells and potential intermediate progenitors. Until now, no marker specific for MaSCs has been identified. On the other hand, the existence of multipotent MaSCs in adults remains in debate as lineage-tracing studies using the pan-basal markers keratin 5 (K5; also known as Krt5) and K14 generate controversial results^{3,4}. Multipotent MaSCs may have been missed in other basal subpopulation lineage tracing studies using Lgr5 or Axin2, and the rare occurrence of clones containing both lineages (bi-lineage) could be due to the periodic luminal expression of these genes^{3,5,6}. Here we show that Procr, a novel Wnt target in the mammary gland, marks a unique population of multipotent MaSCs.

Wnt signalling is instrumental for MaSC self-renewal^{5,7,8}. Our previous work demonstrated that the Wnt3A protein can expand MaSCs in three-dimensional Matrigel culture and maintain their stem cell properties⁷. Taking advantage of this in vitro system, we performed microarray analysis of the cultured MaSCs in an attempt to identify Wnt targets specifically expressed in MaSCs (FIG. 5a). Among the candidates whose expression was increased in the presence of Wnt3A, we identified Procr (FIG. 5a).

Quantitative polymerase chain reaction (qPCR) confirmed that the gene is upregulated by Wnt3A treatment (FIG. 5b).

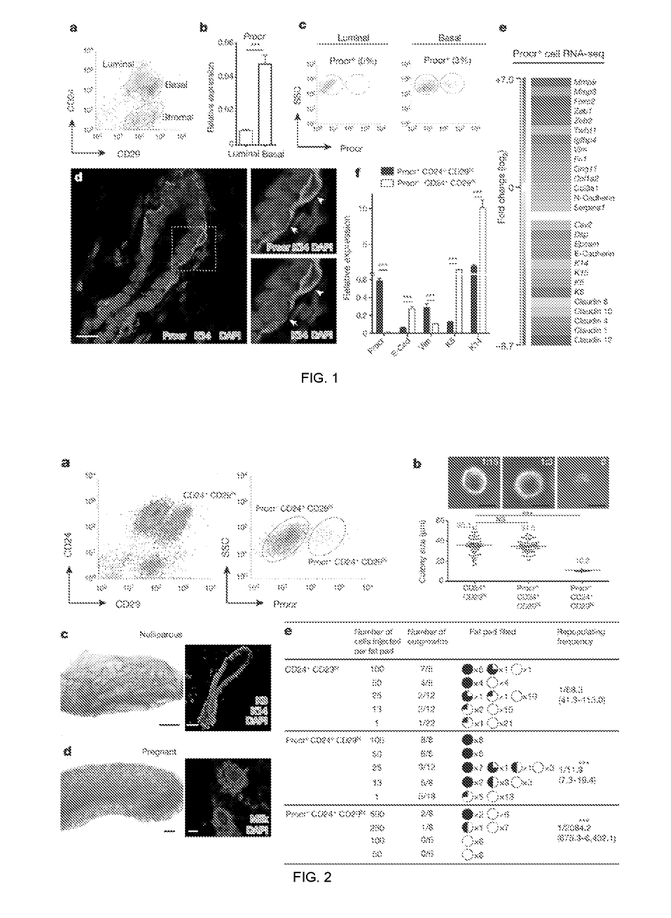

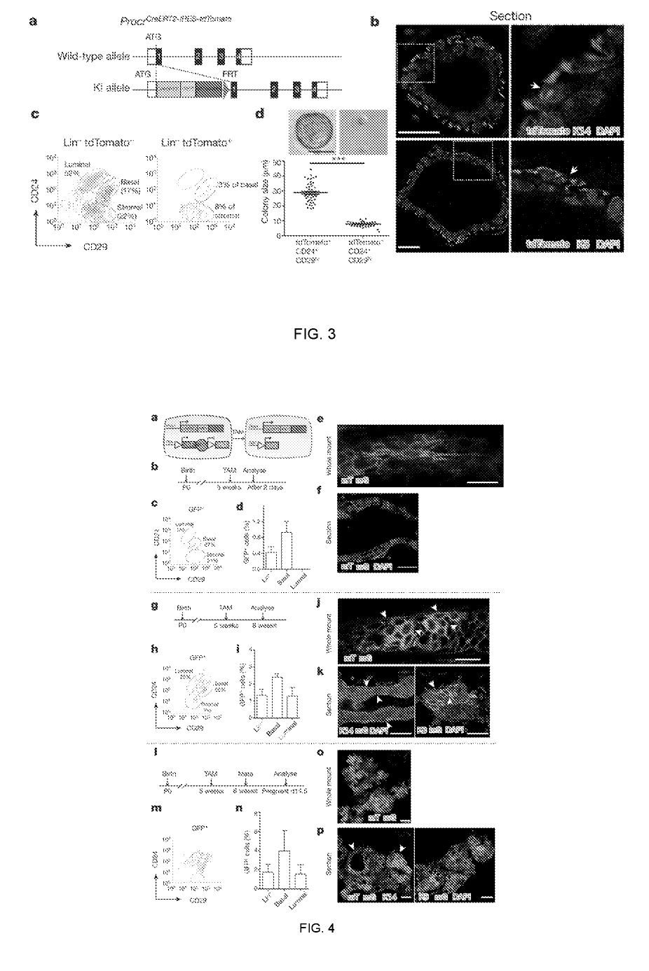

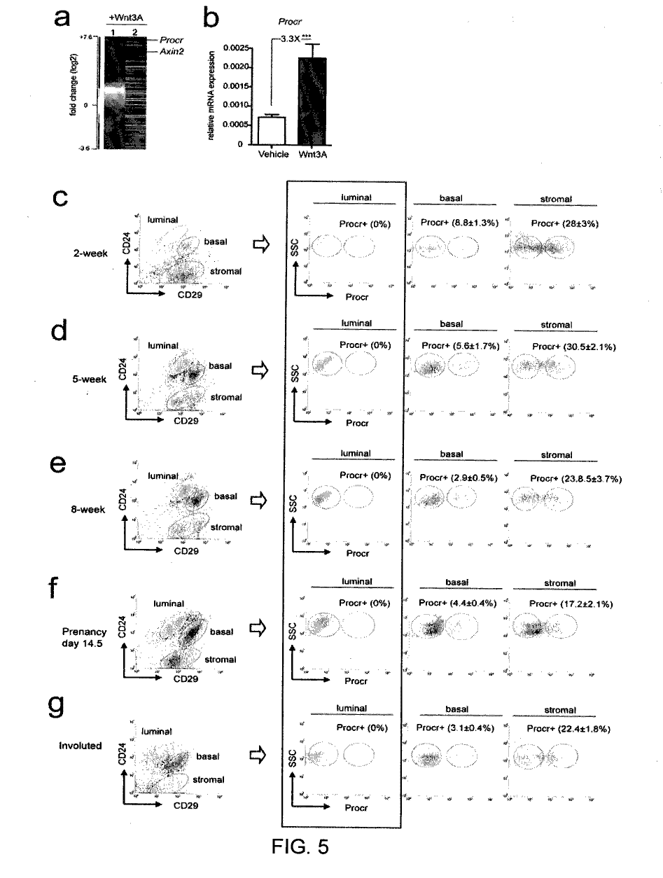

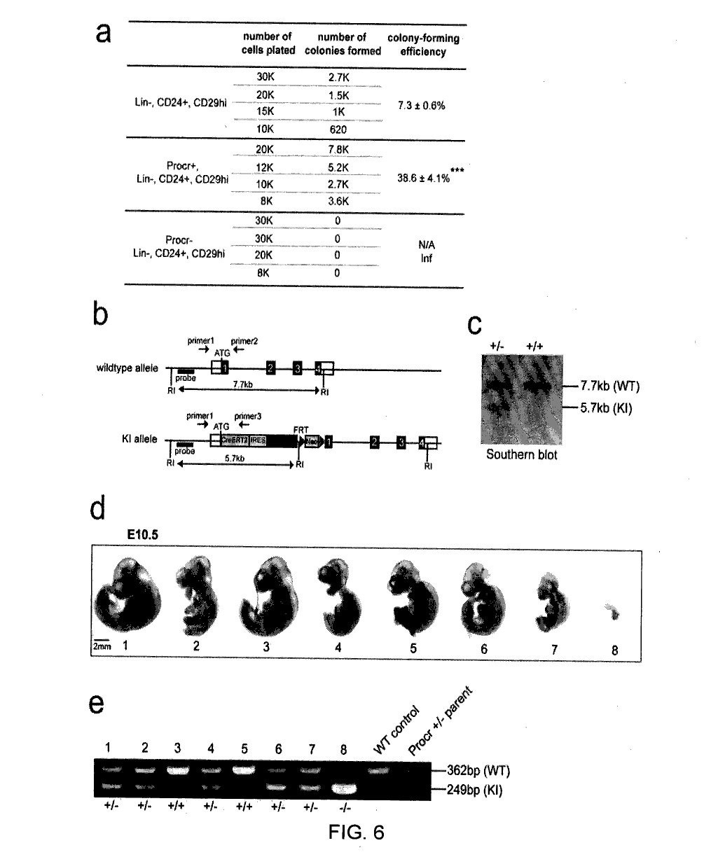

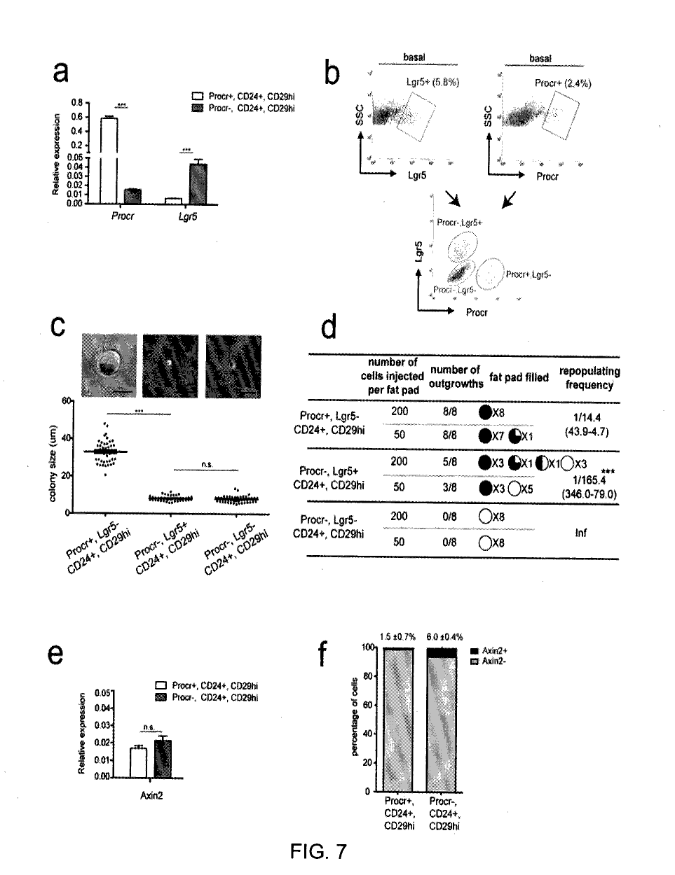

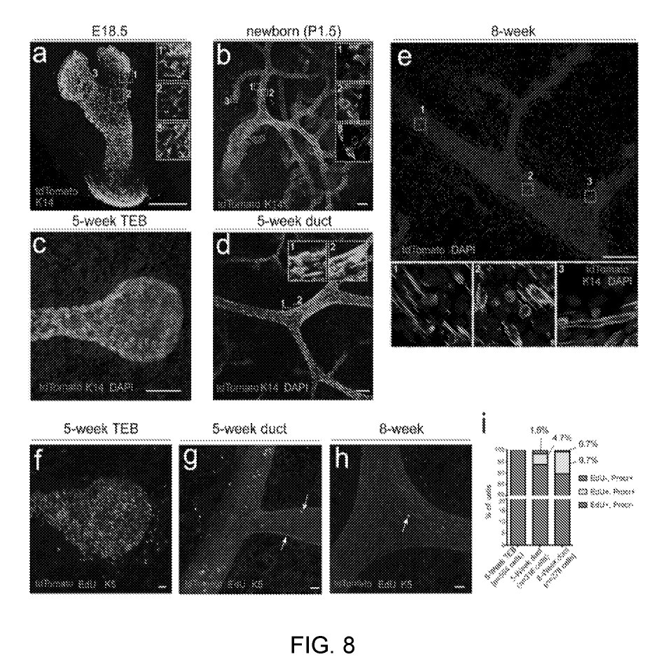

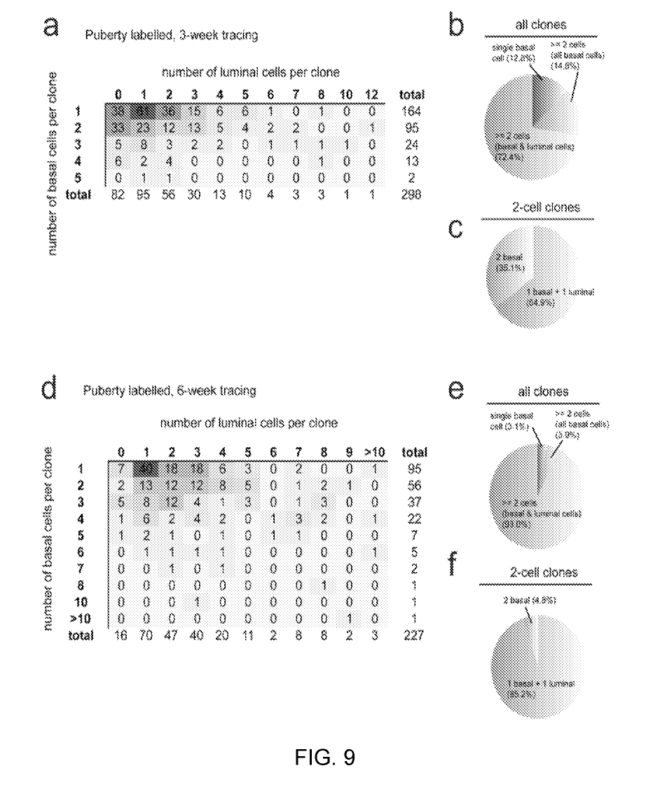

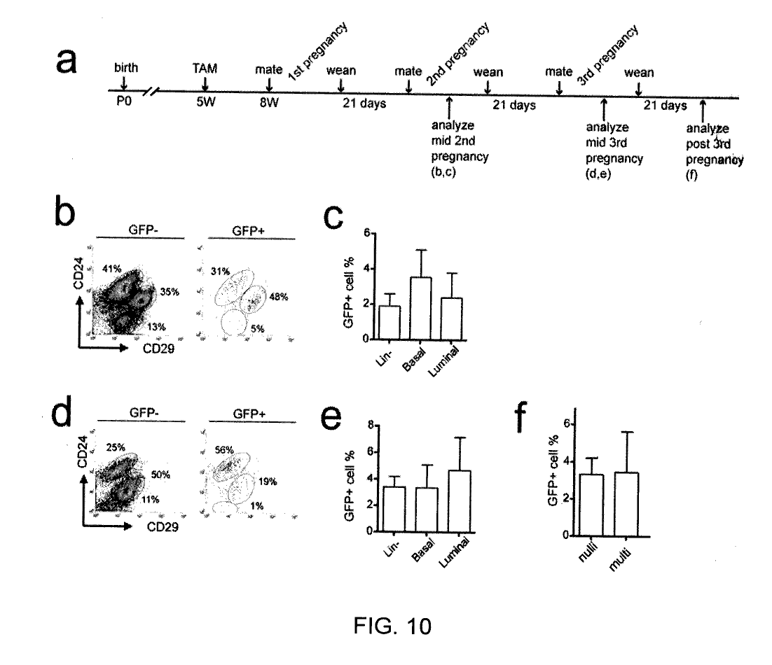

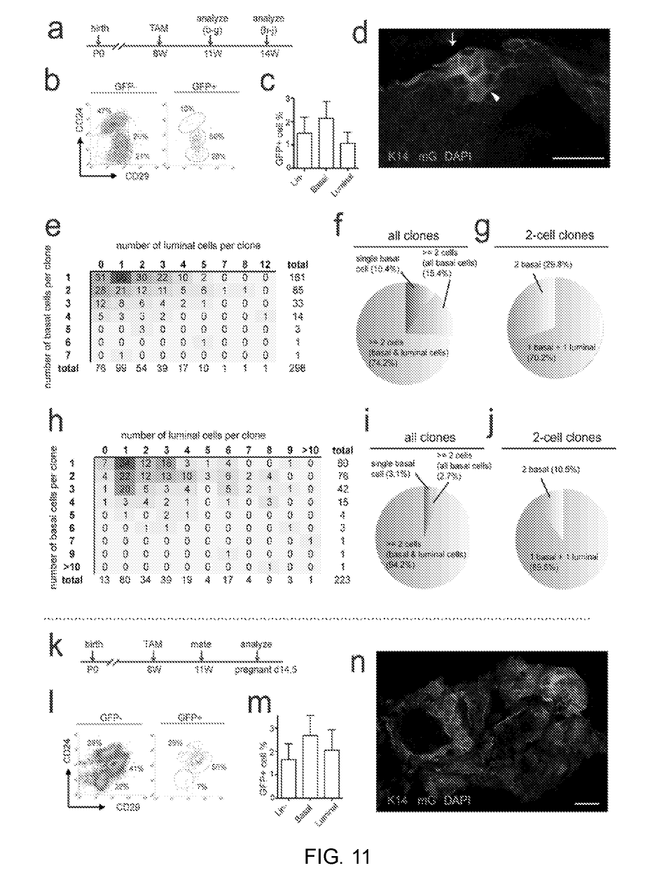

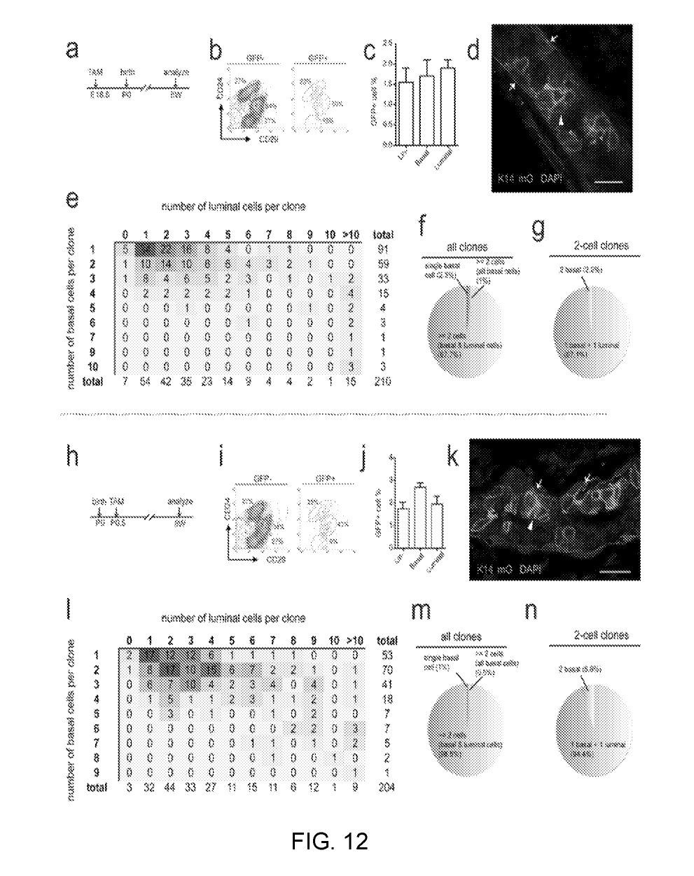

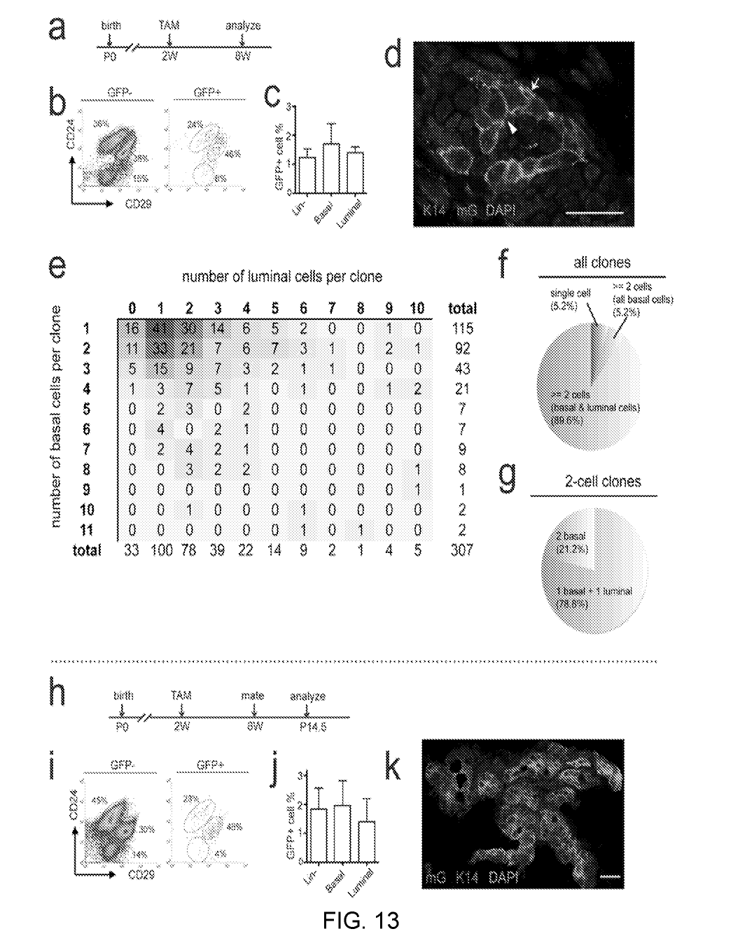

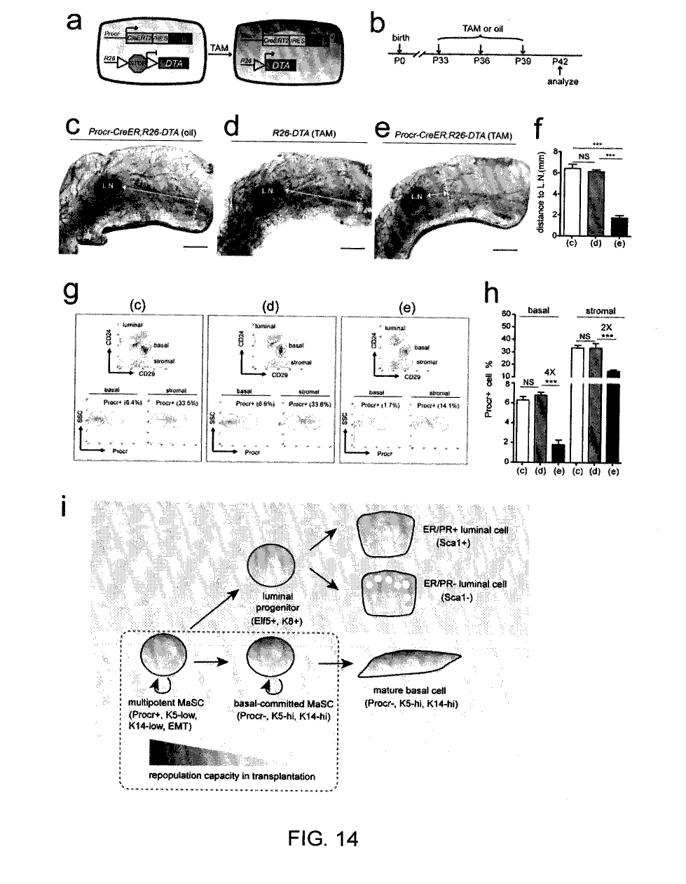

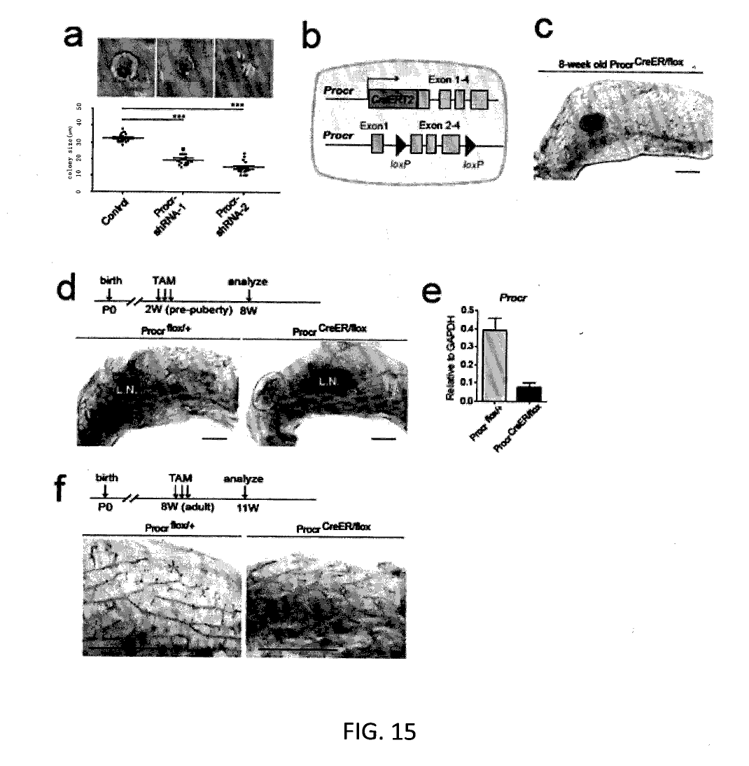

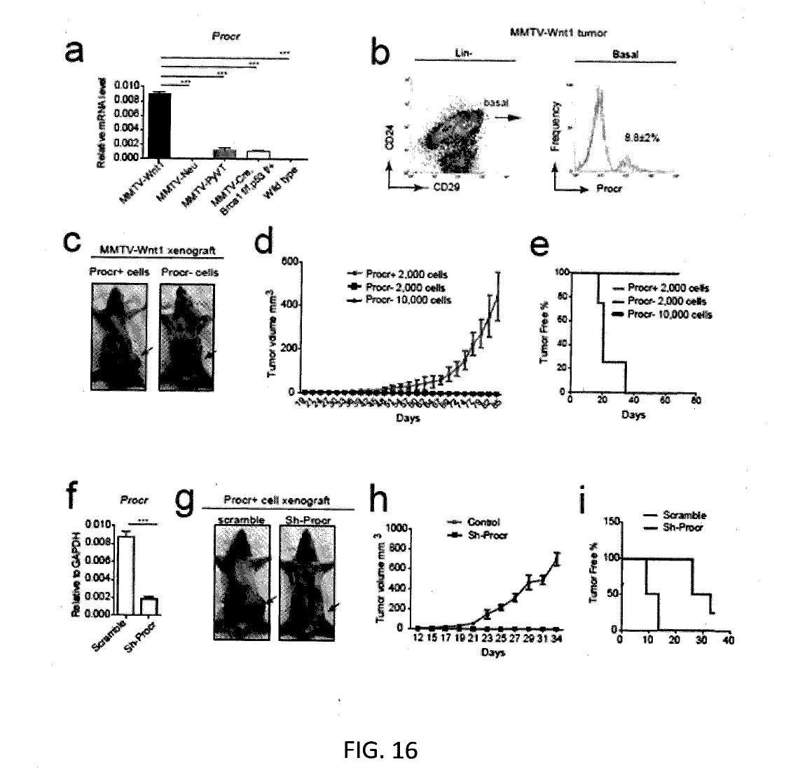

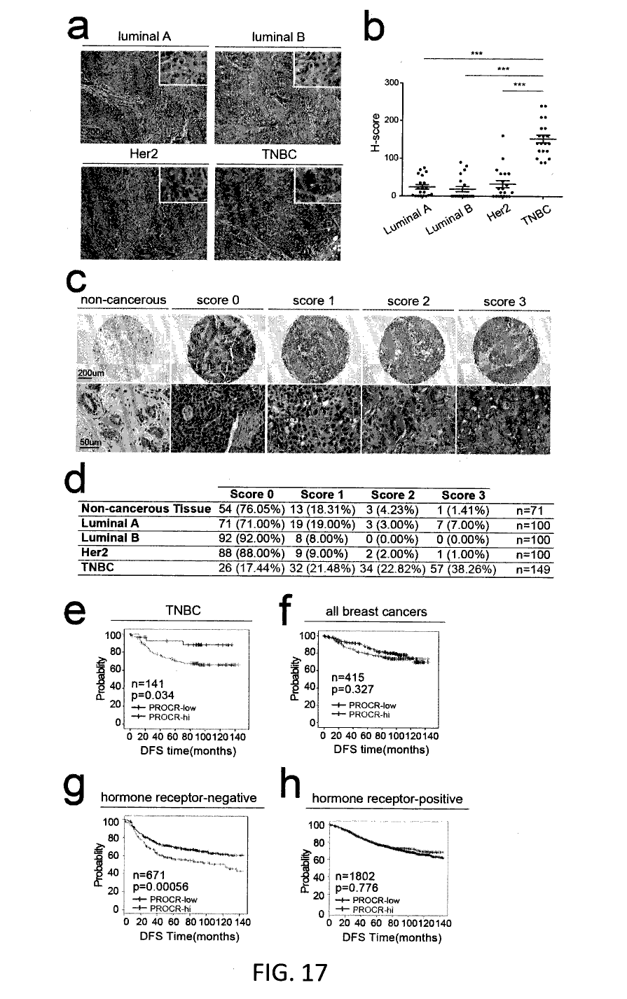

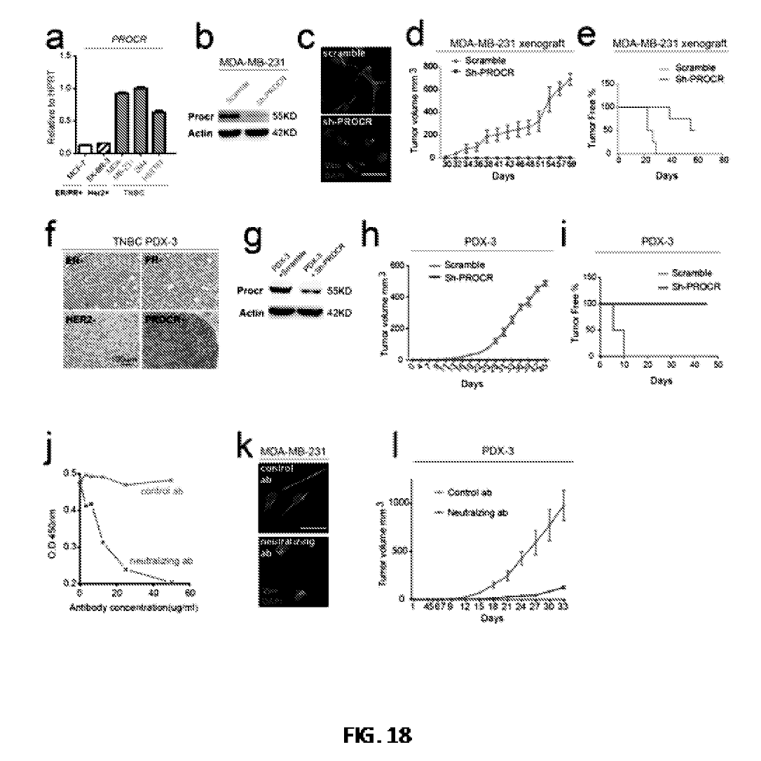

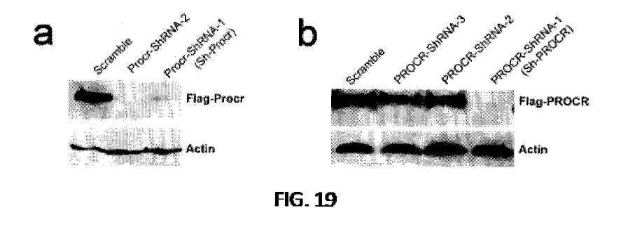

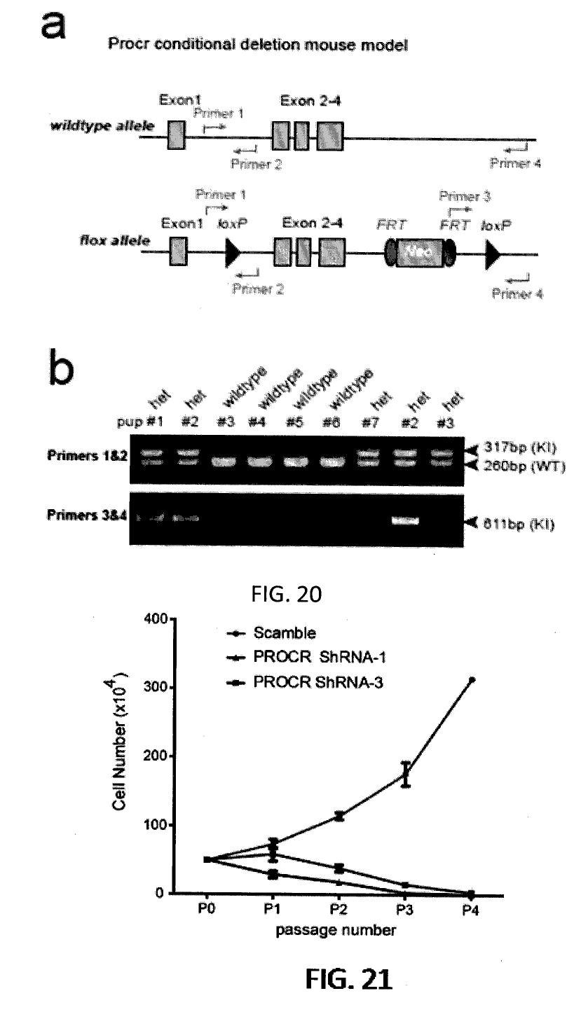

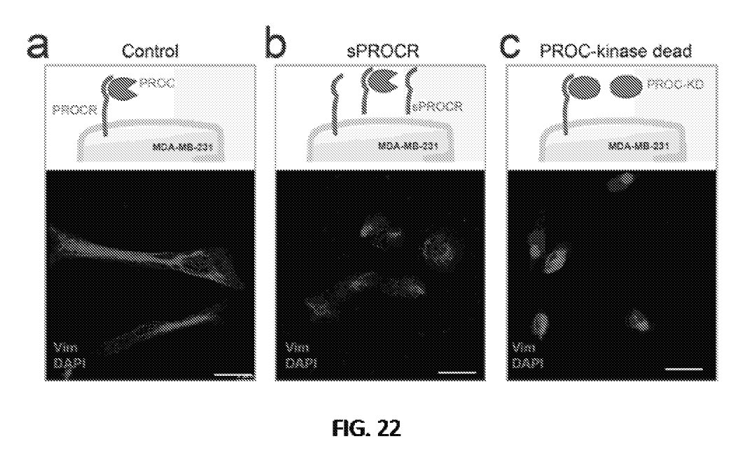

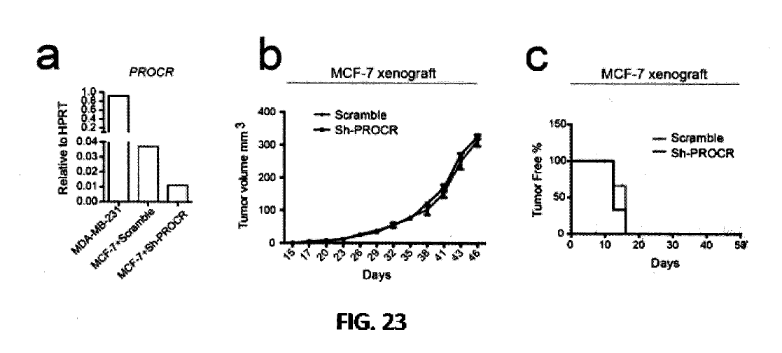

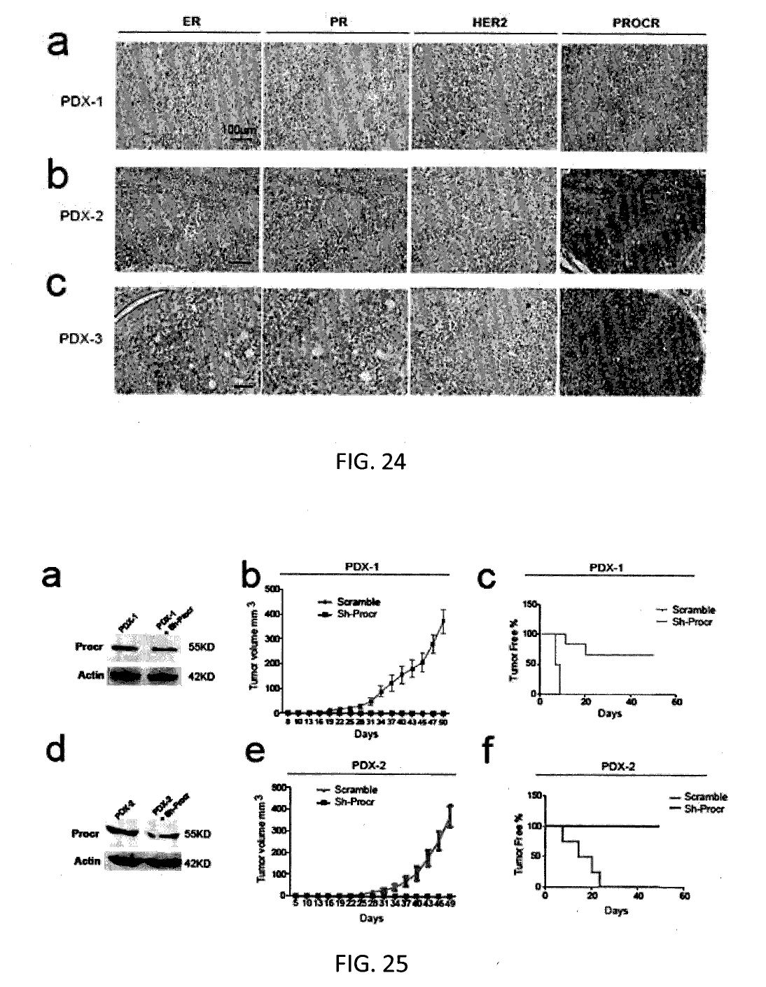

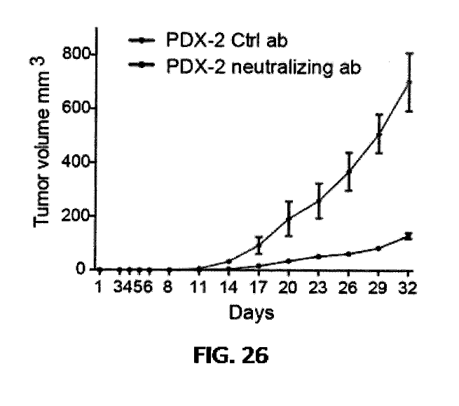

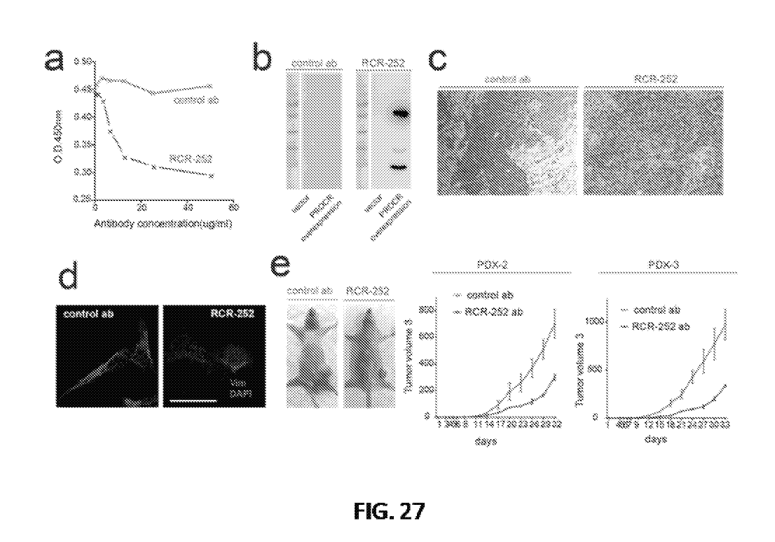

Procr is a single-pass transmembrane protein originally recognized as protein C receptor through its roles in anticoagulation, inflammation and haematopoiesis⁹⁻¹⁴. We investigated whether Procr is normally expressed in the mammary epithelium. We isolated basal (Lin⁻CD24⁺CD29^{hi}) and luminal (Lin⁻CD24⁺CD29^{lo} cells from 8-week-old virgin mammary glands (FIG. 1a), and found that Procr is expressed at higher levels in basal cells (FIG. 1b). Furthermore, fluorescence-activated cell sorting (FACS) analysis indicated that Procr labels 3-7% of basal cells depending on the genetic background (about 2.9±0.5% in CD1 and 7±1.5% in B6), while Procr+ cells were not found among luminal cells (FIG. 1c and FIG. 5c-g). Procr⁺ cells were also detected in the stromal cell compartment (FIG. 5c-g). Notably, the Procr expression patterns were similar throughout development (FIG. 5c-g). Immunostaining confirmed that a subpopulation of basal cells expresses Procr (FIG. 1d). Intriguingly, Procr⁺ cells appeared to express less K14 in comparison to their neighbouring Procr− basal cells (FIG. 1d). Next, we isolated Procr⁺ and Procr− cells from the basal cell population and performed RNA-sequencing (RNA-seq) analysis. We found that basal Procr⁺ cells exhibit features of epithelial-to-mesenchymal transition (EMT), with lower expression of epithelial signatures, for example, Epcam, E-cadherin and claudins, and with increased expression of mesenchymal signature genes, for example, Vim, N-cadherin (also known as Cdh2), Foxc2, Zeb1 and Zeb2 (FIG. 1e). Of note, the basal keratins K5 and K14 were expressed at lower levels in Procr+ cells compared with Procr− basal cells (FIG. 1e). These observations were confirmed by qPCR analysis (FIG. 1f).

We next examined the behaviours of Procr+ basal cells in vitro and in transplantation assays. We isolated total basal cells (CD24+ CD29^{hi}), Procr+ basal cells (Procr+ CD24+ CD29^{hi}) and Procr− basal cells (Procr− CD24+ CD29^{hi}), and compared their colony-forming ability in three dimensional Matrigel culture as previously described7 (FIG. 2a, b). We found that the enrichment of Procr+ cells increased colony-forming efficiency by five-fold when compared to the total basal cell group. One colony formed out of 15 plated total basal cells, while one colony formed out of three plated Procr+ basal cells (FIG. 2b and FIG. 6a). Colony sizes were indistinguishable between the two groups (FIG. 2b). In striking contrast, Procr− basal cells were not able to form colonies in Matrigel culture, suggesting that MaSCs that have colony-forming abilities were absent from this group.

To assess their mammary gland reconstitution capacity, the three groups of isolated cells were transplanted into cleared fat pads. We found that Procr+ basal cells generate the mammary gland more efficiently (repopulating frequency of 1/12) than total basal cells (1/68) (FIG. 2e). The outgrowths displayed normal morphology and marker expression (FIG. 2c). When recipient mice were in late pregnancy, the mammary gland resulting from the transplanted Procr+ basal cells consisted of a dense ductal system ending in clusters of milk-producing alveoli (FIG. 2d). In contrast, Procr− basal cells showed markedly lower stem cell frequency (1/2,084) (FIG. 2e). These findings demonstrate that the CD24+ CD29^{hi} basal population can be further enriched for MaSCs using the marker Procr.

We found that Lgr5+ cells fell into the Procr− population that has drastically reduced regenerative capability (FIG. 7a, b), raising the question as to whether Lgr5+ cells are enriched for MaSCs. To address this, we isolated the three subpopulations of basal cells, Procr+ Lgr5−, Procr− Lgr5+ and Procr− Lgr5− and examined their regenerative capacities. Consistent with our earlier results, the Procr+ Lgr5− cells efficiently formed colonies in vitro and readily reconstituted mammary gland in transplantation (repopulating frequency of 1/14) (FIG. 7c, d). Procr− Lgr5+ cells were not able to form colonies in vitro. Interestingly, they were able to reconstitute in vivo by transplantation, although with a significantly lower repopulating frequency (1/165) (FIG. 7c, d). Considering a repopulating frequency of 1/68 for total basal cells, our results indicated that Lgr5 expression was not enriched in regenerative MaSCs. This conclusion is different from a previous report¹⁵, yet is consistent with two other studies^{4,6}. Finally, the Procr− Lgr5− cells were depleted of MaSCs and failed to regenerate in vitro or in vivo (FIG. 7c, d).

We next investigated whether the population of Procr+cells behave as multipotent MaSCs under physiological conditions. To this end, we generated a knock-in allele of Procr by integrating a CreERT2-IREStdTomato cassette at the first ATG codon (FIG. 3a and FIG. 6b, c). Heterozygous mice were healthy and fertile. Homozygotes died before embryonic day (E) 10.5 (FIG. 6d, e), resembling the Procr-null mutant mice ¹⁶. Confocal imaging of histological sections indicated that tdTomato+ cells resided in the basal layer yet expressed lower levels of K5 and K14 compared with tdTomato− basal cells (FIG. 3b). FACS analysis indicated that 3% of basal cells were tdTomato+ and no tdTomato+ cells were found in luminal cells. Reminiscent of the expression of Procr itself, some tdTomato+ cells were present in stromal cells (FIG. 3c).We isolated tdTomato+ and tdTomato− cells from the basal group and assessed their colony formation capability. We found that tdTomato+ cells form colonies efficiently in vitro, whereas tdTomato31 cells cannot (FIG. 3d). These results demonstrate that the Procr^{CreERT2-IRES-tdTomato} allele faithfully recapitulates endogenous Procr expression.

The generation of the Procr^{CreERT2-IRES-tdTomato} mouse allowed us to examine the expression of Procr+ cells in detail. Using whole-mount confocal imaging analysis, Procr+cells were identified as being sparsely located in E18.5 and newborn (postnatal day (P) 1.5) mammary gland. At these stages, before the formation of the terminal end buds (TEBs), Procr+ cells could be detected in the middle or at the tip of the mammary ducts (FIG. 8a, b). In puberty, dispersed individual Procr+ cells were predominantly present in the mammary ducts, whereas no Procr+ cells were detected in the TEBs (FIG. 8c, d). In the mature mammary gland, individual Procr+cells were also located over the ducts (FIG. 8e). As the TEB is the most proliferative structure in the pubertal gland, our observations suggest that Procr+ cells are not the major proliferative force, consistent with the properties of stem cells rather than transient amplifying cells. By 5-ethymyl-29-deoxyuridine (EdU) incorporation assays, we found that in pubertal or mature mammary glands, the majority of Procr+ cells indeed enter the cell cycle (FIG. 8f-8i). The seemingly higher percentage of EdU+ Procr+ population cells in mature ducts is probably due to a lower number of EdU+ cells at this stage (FIG. 8i). Our data suggest that Procr+ cells are proliferative cells residing in the mammary ducts.

To trace the fate of Procr+ cells, we crossed the Procr^{CreERT2-IRES-tdTomato/+} allele with the ^{Rosa26mTmG/+} (R26^{mTmG/1}) reporter strain¹⁷ (FIG. 4a). We first tracked the developmental fate of Procr+ cells in postnatal mammary glands by administering tamoxifen to Procr^{CreERT2/1}; R26^{mTmG/1} pubertal mice (5 weeks old) and analysing the contribution of labelled cells to the mature epithelial network once the mice had reached adulthood. Expression of green fluorescent protein (GFP) was not observed in un-induced mice (data not shown). Short time tracing (48 h) and confocal whole-mount imaging allowed us to visualize single elongated cells initially labelled by GFP (FIG. 4b, 4e). Immunostaining in tissue sections confirmed that the initially GFP+ cells were basal cells (FIG. 4f). Quantification of the labelling events by FACS analysis indicated that no luminal cells are labelled at the beginning of the analysis (FIG. 4c, 4d). There were some labelled stromal fibroblasts, which also express Procr (FIG. 4c, 4d). After 3 weeks of tracing, the GFP+ cells expanded in number (FIG. 4g-4i). In addition, their pattern extended to include luminal cells. Clonal expansion of GFP+ cells was visualized by whole-mount imaging, and the clones consisted of both elongated and columnar cells (FIG. 4j). Immunostaining confirmed that GFP+ cells are present in both basal and luminal layers (FIG. 4k). Clonal analysis revealed that the majority of the clones (72%) are bi-lineage, giving rise to basal and luminal cells. Notably, about 13% of the clones were single basal cells that had not entered division since labelling; no luminal clones were found (FIGS. 9a, 9b). Importantly, the majority of two-cell clones (65%) comprised one luminal cell and one basal cell (FIG. 9c). When the tracing was prolonged to 6 weeks, the average clone sizes increased over time, and the proportion of the bi-lineage clone also increased (from 72% to 93%), indicating that more basal cells had differentiated into luminal cells (FIGS. 9d, 9e). The percentage of bi-lineage two-cell clones increased (from 65% to 85%), suggesting that more initially GFP+ labelled cells had asymmetrically divided to become luminal cells (FIG. 90. During pregnancy, GFP+ cells contributed to alveolus formation (FIG. 4l-4o). One alveolus could consist solely of GFP+ cells or harbour both GFP+ and mTomato+ cells (FIG. 4p), indicating that alveoli can originate from one or more progenitor(s), which is consistent with previous reports^{4,5}. GFP+ cells were maintained at similar percentages across multiple pregnancies, showing that Procr+ cells are capable of long-term self-renewal (FIGS. 10a-10f). Notably, GFP+ stromal cells did not expand over tracing, suggesting a less proliferative nature of Procr+ fibroblasts (FIGS. 4c, 4h, 4m and FIG. 10b, 10d).

The multipotency of Procr+ basal cells was examined by initiating the labelling in 8-week-old adult mice (FIG. 11a). After 3 weeks of tracing, the majority of labelled cells differentiated into bi-lineage clones (74%) (FIG. 11b-11g), and the percentage increased to 94% by 6 weeks (FIG. 11h-11j). From 3 weeks to 6 weeks, the percentage of bi-lineage two-cell clones also increased (from 70% to 90%). Upon pregnancy, GFP+ cells differentiated to form alveoli (FIG. 11k-11n).

We next investigated the contribution of Procr+ cells to early mammary development by initiating the labelling in E18.5, P0.5 and prepubescent mice (2 weeks old) (FIGS. 12, 13) and analyzing the contribution of GFP+ cells in mature adults. By FACS analysis and immunostaining, GFP+ cells were found in both basal and luminal populations. Eight-week tracing from late embryo or at birth predominantly led to bi-lineage clones in adults (98% and 99%) (FIG. 12f, m). Consistently, 6-week tracing of Procr+ cells in prepubescent mice (2 weeks old) mostly resulted in bi-lineage clones (90%) (FIG. 13f). During pregnancy, GFP+ cells contributed to alveoli formation (FIG. 13h-13k). Taken together, these lineage tracing experiments initiated in the embryonic and various postnatal stages show that Procr+ cells contribute to both basal and luminal cell lineages.

To investigate the physiological requirement of Procr+ cells in mammary gland development, we performed targeted ablation of these cells in developing mammary glands. We generated the Procr^{CreERT2/+}; R26^{DTA/+} strain to conditionally express diphtheria toxin (DTA) in Procr+ cells (FIG. 14a). We administered tamoxifen in Procr^{CreERT2/+}; R26^{DTA/+} pubertal mice at P33 every 3 days, and evaluated the effects of targeted ablation of Procr+ cells 9 days later (FIG. 14b). At this stage, both the oil-treated control mammary epithelium and the tamoxifen-treated R26^{DTA/1} control mammary epithelium had grown to the distal edge of the fat pad (FIG. 14c, 14d). In striking contrast, tamoxifen administration in Procr^{CreERT2/+}; R26^{DTA/+} mice largely prevented the growth of the epithelium (FIG. 14e, 14f). FACS analysis indicated that the basal Procr+ cells were efficiently ablated (FIG. 14g, 14h). Together, these results suggest that the Procr+ cells are important for the development and maintenance of adult mammary gland.

Our study identifies Procr as a novel Wnt target in the mammary epithelium. Procr+ cells express lower levels of K5/K14 compared with other basal cells. They are also unique in that they are multipotent by lineage tracing, and show the highest repopulation efficiency by transplantation. Such a population of cells has not been described before. Much effort has been devoted to delineating the relationships between different epithelial cell populations in the mammary gland. Our work suggests that Procr+ cells are at the top of the hierarchy, supporting the model that multipotent and unipotent stem cells coexist in the adult mammary gland, reconciling the differences found between previous lineage tracing and transplantation studies (FIG. 14i).

EMT has been linked to the stemness properties of cancer cells¹⁸. As Procr+ MaSCs exhibit EMT signatures in the normal mammary gland, it is tempting to speculate that Procr+ MaSCs represent one of the origins of breast cancer stem cells. Indeed, in human breast cancer, Procr is expressed in the CD44+ (cancer-stem-cell-enriched) group¹⁹. Procr expression in cancer cell lines promotes tumour formation^{20,21} and metastasis^{22,23.} More similarities may exist between normal stem cells and malignant stem cells.

### Methods

Experimental animals. To generate mice expressing CreERT2-IRES-tdTomato under control of the endogenous Procr promoter, we generated the targeting construct depicted in FIG. 3a and FIG. 6b. Female mice of Rosa26^{mTmG/-}, Rosa26^{DTA/+} (Jackson Laboratories), Axin2^{lacZ/+} (ref 24), Lgr5^{eGFP-IRES-CreERT2/+} (Lgr5-GFP) (ref 25), CD1, B6 and Nude strains were used in this study. For lineage tracing experiments induced in prepubescent, pubertal and mature adult mice, animals received a single intraperitoneal injection of 4 mg per 25 g body weight of tamoxifen (TAM; Sigma-Aldrich) diluted in sunflower oil. For lineage tracing experiments induced at birth, each mouse received a single injection of 125 mg tamoxifen. To induce recombination in embryos, pregnant mothers at day 18.5 were injected with a single dose of 0.5 mg per 25 g body weight of tamoxifen. For DTA-mediated cell ablation experiments, pubertal mice were injected with 4 mg per 25 g bodyweight of tamoxifen in sunflower oil every 3 days a total of three times. Experimental procedures were approved by the Animal Care and Use Committee of Shanghai Institute of Biochemistry and Cell Biology, Chinese Academy of Sciences.

Quantification of lineage-specific cells and the size of clones. A minimum of three different mice were analysed per condition. Dissociated single mammary cells were FACS analysed for the GFP+ cells proportion in basal and luminal compartments. A minimum of 3 mice were analysed by FACS analysis and a minimum of 20 sections were analysed by immunohistochemistry of K14 and K8 to discern the basal and luminal composition of GFP+ cells. Representative clones were documented by confocal imaging. For clonal analysis, a minimum of 200 GFP+ clones were analysed per time point. For each clone, the number of cells and their K14 or K8 expression were scored. The clones were grouped in three classes: one-cell, two-cell, and clones with more than two cells.

Antibodies. Antibodies used were: rat anti-Procr (1:50, eBioscience, catalogue #13-2012, clone 1560), rat anti-K8 (1:250, Developmental Hybridoma Bank, TROMA-I), rabbit anti-K14 (1:1,000, Covance), rabbit anti-KS (1:1000, Covance), rabbit anti-milk (1:500, Nordic Immunological Laboratories).

Primary cell preparation. Mammary glands from 8- to 12-week-old virgin or an otherwise specified stage of female mice were isolated. The minced tissue was placed in culture medium (RPMI 1640 with 25 mM HEPES, 5% fetal bovine serum, 1% penicillin-streptomycin-glutamine (PSQ), 300 U ml⁻¹ collagenase III (Worthington)) and digested for 2 h at 37° C. After lysis of the red blood cells in NH₄Cl, a single-cell suspension was obtained by sequential incubation with 0.25% trypsin-EDTA at 37° C. for 5 min and 0.1 mg ml⁻¹ DNase I (Sigma) for 5 min with gentle pipetting, followed by filtration through 70 μm cell strainers.

Cell labelling and flow cytometry. The following antibodies in 1:200 dilutions were used: biotinylated and FITC-conjugated CD31, CD45, TER119 (BD PharMingen, clone MEC 13.3, 30-F11 and TER-119; catalogue # 553371, #55307, # 553672, # 553372, # 553080 and # 557915), CD24-PE/cy7, CD29-APC (Biolegend, clone M1/69 and HMb1-1; catalogue #101822 and #102216), Procr-PE (eBioscience, clone 1560, catalogue #12-2012), Streptavidin-V450, and Streptavidin-FITC (BD PharMingen). Antibody incubation was performed on ice for 15 min in HBSS with 10% fetal bovine serum. All sortings were performed using a FCASJazz (Becton Dickinson). The purity of sorted population was routinely checked and ensured to be more than 95%.

In vitro colony formation assay. FACS-sorted cells were resuspended at a density of 4×10⁵ cells ml⁻¹ in chilled 100% growth-factor-reducedMatrigel (BD Bioscience), and the mixture was allowed to polymerize before covering with culture medium (DMEM/F12, ITS (1:100; Sigma), 50 ng ml⁻¹ EGF, plus either vehicle (1% CHAPS in PBS) or 200 ng ml⁻¹ Wnt3A²⁶). Culture medium was changed every 24 h. Primary colony numbers were scored after 6-7 days in culture. The colonies were mostly spherical. In cases that colonies were oval, the long axis was measured.

RNA extraction, microarray and RNA sequencing. For microarray, total RNA from second-passage MaSC colonies cultured in the presence of vehicle andWnt3A was extracted with PicoPure (Arcturs) in accordance with the manufacturer's protocol. At the second passage, MaSC colonies in Wnt3A treatment can efficiently reconstitute new mammary glands in transplantation assays, an indication of retaining stemness, while MaSC colonies in vehicle have completely lost the reconstitution capabilities⁷. RNA concentration was determined with NanoDrop ND-1000, and quality was determined using the RNA 6000 Nano assay on the Agilent 2100 Bioanalyzer (Agilent Technologies). Affymetrix microarray analysis, fragmentation of RNA, labelling, hybridization to Mouse Genome 430 2.0 microarrays and scanning were performed in accordance with the manufacturer's protocol (Affymetrix). For RNA-seq, total RNA from freshly isolated Lin− CD24+ CD29^{hi} Procr+ cells and Lin− CD24+ CD29^{hi} Procr− cells were extracted with Trizol. RNAseq libraries were prepared according to the manufacturer's instructions (Illumina) and then applied to sequencing on Illumina HiSeq 2000 in the CAS-MPG Partner Institute for Computational Biology Omics Core, Shanghai. In total, around 32 million 1×100 single reads for each sample were obtained and uniquely mapped to the mm9 mouse genome with more than 70% mapping rate for both samples using TopHat 1.3.3. Differential gene expression analysis was carried out using Cuffdiff 2.0.2, and genes with significant alteration were extracted for a further analysis.

EdU labelling. In vivo EdU labelling was accomplished by intraperitoneal injections of EdU (0.2 mg per 10 g body weight) followed by harvest 3 h after injection. Samples were subjected to Click-it chemistry (Invitrogen).

Immunohistochemistry. Whole-mount staining was performed as described previously⁴. Frozen sections were prepared by air-drying and fixation for 1 h in cold MeOH or PFA. Tissue sections were incubated with primary antibodies at 4° C. overnight, followed by washes, incubation with secondary antibodies for 2 h at 25° C., and counterstaining with DAPI (Vector Laboratories). For all the immunofluorescence staining at least three independent experiments were conducted. Representative images are shown in the figures.

Mammary fat pad transplantation and analysis. Sorted cells were resuspended in 50% Matrigel, PBS with 20% FBS, and 0.04% Trypan Blue (Sigma), and injected in 10 ml volumes into the cleared fat pads of 3-week-old female. Reconstituted mammary glands were harvested after 8-10 weeks post-surgery. Outgrowths were detected by under a dissection microscope (Leica) after Carmine staining. Outgrowths with more than 10% of the host fat pad filled were scored as positive.

Statistical analysis. Student's t-test was performed and the P value was calculated in Prism on data represented by bar charts, which consisted of results from three independent experiments unless specified otherwise. For all experiments with error bars, the standard deviation (s.d.) was calculated to indicate the variation within each experiment. The experiments were not randomized. The investigators were not blinded to allocation during experiments and outcome assessment.

Primers used in qPCR analysis. Primers used were as follows. Procr forward, CTCTCTGGGAAAACTCCTGACA (SEQ ID NO.:5); Procr reverse, CAGGGAGCAGCTAACAGTGA (SEQ ID NO.:6); K5 forward, TCTGCCATCACCCCATCTGT (SEQ ID NO.:7); K5 reverse, CCTCCGCCAGAACTGTAGGA (SEQ ID NO.:8); K14 forward, TGACCATGCAGAACCTCAATGA (SEQ ID NO.:9); K14 reverse, ATTGGCATTGTCCACGG (SEQ ID NO.:10); E-cadherin forward, CAGGTCTCCTCATGGCTTTGC (SEQ ID NO.:11); E-cadherin reverse, CTTCCGAAAAGAAGGCTGTCC (SEQ ID NO.:12); Vim forward, CGTCCACACGCACCTACAG (SEQ ID NO.:13); Vim reverse, GGGGGATGAGGAATAGAGGCT (SEQ ID NO.:14); Lgr5 forward, CCTACTCGAAGACTTACCCAGT (SEQ ID NO.:15); Lgr5 reverse, GCATTGGGGTGAATGATAGCA (SEQ ID NO.:16); Axin2 forward, AGCCTAAAGGTCTTATGTGGCTA (SEQ ID NO.:17); Axin2 reverse, ACCTACGTGATAAGGATTGACT (SEQ ID NO.:18).

### REFERENCES

1. Shackleton, M. et al. Generation of a functional mammary gland from a single stem cell. Nature 439, 84-88 (2006).

2. Stingl, J. et al. Purification and unique properties of mammary epithelial stem cells. Nature 439, 993-997 (2006).

3. Van Keymeulen, A. et al. Distinct stem cells contribute to mammary gland development and maintenance. Nature 479, 189-193 (2011).

4. Rios, A. C., Fu, N. Y., Lindeman, G. J. & Visvader, J. E. In situ identification of bipotent stem cells in the mammary gland. Nature 506, 322-327 (2014).

5. van Amerongen, R., Bowman, A. N. & Nusse, R. Developmental stage and time dictate the fate of Wnt/b-catenin-responsive stem cells in the mammary gland. Cell Stem Cell 11, 387-400 (2012).

6. de Visser, K. E. et al. Developmental stage-specific contribution of LGR51 cells to basal and luminal epithelial lineages in the postnatal mammary gland. J. Pathol. 228, 300-309 (2012).

7. Zeng, Y. A. & Nusse, R. Wnt proteins are self-renewal factors for mammary stem cells and promote their long-term expansion in culture. Cell Stem Cell 6, 568-577 (2010).

8. Badders, N. M. et al. The Wnt receptor, Lrp5, is expressed by mouse mammary stem cells and is required to maintain the basal lineage. PLoS ONE 4, e6594 (2009).

9. Fukudome, K. & Esmon, C. T. Identification, cloning, and regulation of a novel endothelial cell protein C/activated protein C receptor. J. Biol. Chem. 269, 26486-26491 (1994).

10. Cheng, T. et al. Activated protein C blocks p53-mediated apoptosis in ischemic human brain endothelium and is neuroprotective. Nature Med. 9, 338-342 (2003).

11. Balazs, A. B., Fabian, A. J., Esmon, C. T. & Mulligan, R. C. Endothelial protein C receptor (CD201) explicitly identifies hematopoietic stem cells in murine bone marrow. Blood 107, 2317-2321 (2006).

12. Vetrano, S. et al. Unexpected role of anticoagulant protein C in controlling epithelial barrier integrity and intestinal inflammation. Proc. Natl Acad. Sci. USA 108, 19830-19835 (2011).

13. Ivanova, N. B. et al. A stem cell molecular signature. Science 298, 601-604 (2002).

14. Bae, J. S., Yang, L., Manithody, C. & Rezaie, A. R. The ligand occupancy of endothelial protein C receptor switches the protease-activated receptor 1-dependent signaling specificity of thrombin from a permeability-enhancing to a barrier-protective response in endothelial cells. Blood 110, 3909-3916 (2007).

15. Plaks, V. et al. Lgr5-expressing cells are sufficient and necessary for postnatal mammary gland organogenesis. Cell Rep. 3, 70-78 (2013).

16. Gu, J. M. et al. Disruption of the endothelial cell protein C receptor gene in mice causes placental thrombosis and early embryonic lethality. J. Biol. Chem. 277, 43335-43343 (2002).

17. Muzumdar, M. D., Tasic, B., Miyamichi, K., Li, L. & Luo, L. A global double fluorescent Cre reporter mouse. Genesis 45, 593-605 (2007).

18. Mani, S. A. et al. The epithelial-mesenchymal transition generates cells with properties of stem cells. Cell 133,704-715 (2008).

19. Shipitsin, M. et al. Molecular definition of breast tumor heterogeneity. Cancer Cell 11,259-273 (2007).

20. Schaffner, F. et al. Endothelial protein C receptor function in murine and human breast cancer development. PLoS ONE 8, e61071 (2013).

21. Hwang-Verslues, W. W. et al. Multiple lineages of human breast cancer stem/progenitor cells identified by profiling with stem cell markers. PLoS ONE 4, e8377 (2009).

22. Beaulieu, L. M. & Church, F. C. Activated protein C promotes breast cancer cell migration through interactions with EPCR and PAR-1. Exp. Cell Res. 313,677-687 (2007).

23. Spek, C. A. & Arruda, V. R. The protein C pathway in cancer metastasis. Thromb. Res. 129 (suppl. 1), S80-S84 (2012).

24. Lustig, B. et al. Negative feedback loop of Wnt signaling through upregulation of conductin/axin2 in colorectal and liver tumors. Mol. Cell. Biol. 22,1184-1193 (2002).

25. Barker, N. et al. Identification of stem cells in small intestine and colon by marker gene Lgr5. Nature 449,1003-1007 (2007).

26. Willert, K. et al. Wnt proteins are lipid-modified and can act as stem cell growth factors. Nature 423, 448-452 (2003).

27. Shehata, M. et al. Phenotypic and functional characterization of the luminal cell hierarchy of the mammary gland. Breast Cancer Res. 14, R134 (2012).

### EXAMPLE 2

### Protein C Receptor Is A Surface Therapeutic Target For Triple-Negative Breast Cancer

Triple-negative breast cancer (TNBC) is a highly aggressive malignancy with no targeted treatment option^{1,2}. Previous study identifies Protein C Receptor (Procr) as a marker for the bipotent mouse mammary stem cell (MaSC)³. Here we report that Procr represents a novel surface therapeutic target for human TNBC. Deletion of Procr diminishes the function of MaSCs and completely inhibits mouse mammary development. In mouse tumor models, Procr-expressing cells are enriched for tumor-initiating cells, whereas knockdown of Procr inhibits tumor growth. In human breast cancer patient samples, PROCR is highly expressed in TNBC subtype, and associated with poor prognosis. Depletion of PROCR potently inhibits tumor growth in Patient Derived Xenograft (PDX) models and cancerous cell line. Moreover, a neutralizing antibody (such as the commercially available RCR-252 from Abcam and a new antibody prepared according to conventional methods known in the art) targeting PROCR function effectively suppresses TNBC tumor growth, underlining a clinically applicable approach for TNBC treatment. Our findings reveal a key role of PROCR in TNBC tumorigenesis and indicate that targeting this surface marker offers a novel treatment strategy for this notorious subtype of breast cancer.

TNBCs are clinically defined by the lack of expression of estrogen receptor (ER), progesterone receptor (PR), and the absence of amplification or overexpression of HER2². This subtype accounts for 15% to 20% of newly diagnosed breast cancer cases, characterized by aggressive behavior, distinct patterns of metastasis, poor patient survival^{2,4}. Treatment of TNBC patients has been challenging due to the heterogeneity and the absence of well-defined molecular targets. Our recently study identifies Procr as a surface marker for mouse MaSCs (Lin⁻, CD24⁺, CD29^{hi}, Procr⁺)³. Due to the close alignment of the expression profile of stem cell-enriched population with TNBC signature indicated by comparative gene expression studies (Lim et al. 2009; Prat et al. 2010), we set to explore the significance of Procr in the prognosis and therapy of TNBC.

Procr is Critical For Mammary Stem Cells and Mammary Development

Besides serving as a marker, the functional role of Procr in regulating MaSCs remains to be determined. To attenuate the expression of Procr, ShRNAs (namely Sh-Procr) were generated and their knockdown efficacies were validated by Western analyses (FIG. 19a). MaSCs were isolated from 8-week old mouse mammary glands by fluorescent associated cell sorting (FACS) and infected with control or Sh-Procr lentivirus in suspension. The infected MaSCs were cultured in 3D matrigel to allow colony formation. We found that the colony sizes are significantly smaller when Procr expression is inhibited, suggesting that Procr is important for the clonogenicity property of MaSCs (FIG. 15a). To investigate the function of Procr in mammary development, we generated Procr conditional deletion allele (Procr^{flox/+}), with two loxP sites inserted to flank exon 2-4 (FIG. 20 and see Methods for detail). Procr^{flox/+} and homozygote (Procr^{flox/flox}) mice are grossly normal (data not shown). To delete Procr specifically in MaSCs, the Procr^{CreER-IRES-tdTomat/+} mice³, hereafter referred to as Procr^{CreER/+}, were bred to Procr^{flox/+} mice (FIG. 15b). The resulting Procr^{CreER/flox} (cKO) mice developed normally and their mammary glands displayed no discernable phenotypes (FIG. 15c). We administered TAM in cKO and the control (Procr^{flox/+}) pre-pubertal mice at 2-week old every other days for 3 times, and evaluated the effects of Procr deletion at 8-week. At this stage, the TAM-treated control mammary gland had completed the epithelium extension and occupied the whole fat pad. Strikingly, TAM administration in cKO mice completely prevented the growth of the epithelium: the mammary gland had very few branches close to the nipple (FIG. 15d). qPCR analysis confirmed the successful knockout (FIG. 15e). Next, to investigate the impact of Procr in mammary homeostasis, TAM was administered in 8-week old adult mice and mammary glands were analyzed after 3 weeks. The Procr^{CreER/flox} mammary gland exhibited hollow and dilated ducts with reduced side branches, while the control Procr^{flox/+} mammary gland was normal (FIG. 15f). Together, these data suggest that Procr is critical for MaSCs in developing mammary gland and in mammary homeostasis.

Procr-Expressing Cells Are Tumor-Initiating Cells in Mouse Basal-Like Tumors

We next investigated the role of Procr in mammary tumor formation. We examined Procr expression level in four murine mammary tumor models. MMTV-Wnt1 forms tumors that predominantly express basal cell markers, a characteristic of TNBC^{5,6}; MMTV-Neu tumor bares Her2 overexpression and is similar to human luminal subtype^{6,7}, MMTV-Pyvt tumor is clustered close to the clinical luminal B subtype^{6,8}, and MMTV-Cre; Brca1^{f/f}, p53^{-/-} tumor carries Brca1 mutation and is associated with the human basal-like tumor profile^{6,9}. By qPCR analysis, we found that Procr expression is particularly high in MMTV-Wnt 1 tumor, compared to the other three tumors and normal mammary glands (FIG. 16a). This observation is consistent with the notion that Procr is a Wnt-target in mammary cells³, and that MMTV-Wnt1 tumors are hypothesized to origin from stem cell malignancy¹⁰.

Next we investigated the tumor-initiating property of Procr-expressing cells. Procr⁺ cells (Lin⁻, CD24⁺, CD29^{hi}, Procr⁺) and Procr⁻ cells (Lin⁻, CD24⁺, CD29^{hi}, Procr⁻) were FACS-isolated from MMTV-Wnt1 primary tumors and xenografted to fat pads of immunocompromised mice (FIG. 2b). We found that Procr+ cells constituted tumors efficiently, whereas Procr− cells cannot form tumors (FIG. 16c-e). No tumor formation was observed even when the injected Procr⁻ cell amount was increased to 5-fold (FIG. 16d-e). To address whether Procr is required for the tumor formation capability of the labeled cells, we inhibited Procr expression by ShRNA. Procr⁺ cells were FACS-isolated from MMTV-Wnt1 primary tumors and virally infected by ShRNA with GFP tag. The infected cells were sorted using GFP and xenografted to fat pads of recipients. Some infected cells were set aside for validation of the knockdown by qPCR (FIG. 16f). We found that inhibition of Procr drastically attenuates the tumor formation of the engrafted tumor cells, while the tumor cells infected by scramble ShRNA forms tumor potently (FIG. 16g-i). Thus, Procr-expressing cells are enriched for tumor-initiating cells in MMTV-Wnt1 tumor, and that inhibition of Procr diminishes their tumor formation capacity.

PROCR Expression is Positively Correlated With TNBC

Next we investigated the expression of PROCR in clinical breast cancer samples by immunohistochemical staining. A total of 80 breast tumors (20 specimens for each subtype) were analyzed. Scoring was conducted according to the proportion of tumor cells with positive stained (0 to 100) and intensity of the stain (0, negative; 1, weak; 2, moderate; 3, strong), with a maximal score of 300. PROCR expression, with score >100, was predominantly observed in TNBC (FIG. 17a, b). To confirm this finding, we subsequently examined PROCR expression in a larger cohort via tissue microarrays (TMAs) comprising 449 breast tumors and 71 non-cancerous mammary controls. PROCR staining was quantified in parallel by two experienced breast disease pathologists who were blinded to all clinical data. TNBCs exhibited markedly higher prevalence of PROCR positive cases (N=123; 82.6% positive, 17.4% negative) than HER2-positive and ER/PR-positive carcinomas (FIG. 17d). We further investigated the relationship between the clinicopathological characteristics and PROCR expression levels (Table 1). As expected, PROCR expression was tightly associated with ER status (P<0.001) and HER2 status (P<0.001) in breast tumors, but there was no other correlation between Procr levels and other clinical pathological features (Table 1).

TABLE 1
Clinicopathological variables and the expression of
PROCR in the study cohort

| | | | PROCR expression | | | | |
|---|---|---|---|---|---|---|---|
| | Number of | | Low | | High | | P^{a} |
| Variables | patients | | N (%) | | N (%) | | value |
| Total | 443 | | 220 | (49.3) | 223 | (50.3) | |
| Age | | | | | | | |
| ≤50 years | 229 | (51.7) | 111 | (25.1) | 118 | (26.6) | |
| >50 years | 214 | (48.3) | 109 | (24.6) | 105 | (23.7) | |
| Menopausal | | | | | | | 0.141 |
| status | | | | | | | |
| Premenopause | 225 | (50.8) | 104 | (23.5) | 121 | (27.3) | |
| Postmenopause | 218 | (49.2) | 116 | (26.2) | 102 | (23.0) | |
| Tumor size | | | | | | | 0.367 |
| ≤2 cm | 206 | (46.5) | 99 | (22.3) | 107 | (24.1) | |
| >2, ≤5 cm | 208 | (47.0.) | 102 | (23.0) | 106 | (23.9) | |
| >5 cm | 20 | (4.5) | 13 | (2.9) | 7 | (1.58) | |
| Cannot be | 9 | (2.0) | 6 | (1.4) | 3 | (0.1) | |
| measured | | | | | | | |
| Lymph node | | | | | | | 0.323 |
| status | | | | | | | |
| Negative | 248 | (56.0) | 118 | (26.6) | 130 | (29.3) | |
| Positive | 195 | (44.0) | 102 | (23.0) | 93 | (21.0) | |
| TNM Stage | | | | | | | 0.400 |
| I | 136 | (30.7) | 61 | (13.8) | 75 | (16.9) | |
| II | 243 | (54.9) | 123 | (27.8) | 120 | (27.1) | |
| III | 49 | (11.1) | 27 | (6.1) | 22 | (5.0) | |
| IV | 2 | (0.5) | 2 | (0.5) | 0 | (0.0) | |
| Unknown | 13 | (2.9) | 7 | (1.6) | 6 | (1.4) | |
| Grade | | | | | | | 0.503 |
| 1 | 7 | (1.6) | 5 | (1.1) | 2 | (0.5) | |
| 2 | 240 | (54.2) | 115 | (26.0) | 125 | (28.2) | |
| 3 | 113 | (25.5) | 55 | (12.4) | 58 | (13.1) | |
| Unknown | 83 | (18.7) | 45 | (10.2) | 38 | (8.6) | |
| ER status | | | | | | | 0.000 |
| Negative | 251 | (56.7) | 104 | (23.5) | 147 | (33.2) | |
| Positive | 192 | (43.3) | 116 | (26.2) | 76 | (17.2) | |
| PR status | | | | | | | 0.439 |
| Negative | 293 | (66.4) | 141 | (32.0) | 152 | (34.5) | |
| Positive | 148 | (33.6) | 77 | (17.5) | 71 | (16.1) | |
| HER-2/neu | | | | | | | 0.000 |
| status | | | | | | | |
| Negative | 247 | (55.8) | 76 | (17.2) | 171 | (38.6) | |
| Positive | 196 | (44.2) | 144 | (32.5) | 52 | (11.7) | |
| Subtype^{b} | | | | | | | 0.000 |
| Luminal | 204 | (46.0) | 122 | (27.5) | 82 | (18.5) | |
| HER-2 | 90 | (20.3) | 67 | (15.1) | 23 | (5.2) | |
| Enrichment | | | | | | | |
| TNBC | 149 | (33.6) | 31 | (7.0) | 118 | (26.6) | |

Abbreviations:
PROCR, protein C receptor;
ER, estrogen receptor;
PR, progesterone receptor;
HER-2, human epidermal growth factor receptor 2;
TNBC, triple negative breast cancer
^{a}Based on Pearson χ2 test (Fisher exact test was used when needed).
^{b}Definition of subtypes: Luminal (ER and/or PR positive), HER-2 Enrichment (ER and PR negative, HER-2 positive), and TNBC (ER negative, PR negative, and HER-2 negative)

To assess the clinical significance of PROCR overexpression, we analyzed the relationship between PROCR levels and disease-free survival (DFS). Among 449 breast cancer cases, the clinical outcomes of 415 cases were obtained. Kaplan-Meier analysis suggested that Procr positivity correlated with poor DFS in TNBC cases (P=0.0341; FIG. 17e), whereas no significant association was found PROCR expression and disease events in the cohort without stratification of molecular subtypes (P=0.327; FIG. 17f). In accordance with our results, the analysis derived from a large public clinical database of breast cancer (Kaplan-Meier Plotter) provided additional support that high levels of PROCR expression correlated with a more poor DFS in patients with hormone-receptor negative breast cancer (P=0.0006, FIG. 17g), whereas PROCR expression has no prognostic value in hormone-receptor positive patients (P=0.776, FIG. 17h). Additionally, both univariate and adjusted multivariate survival analyses suggested a difference between the PROCR-high and PROCR-low groups in TNBCs. Elevated PROCR expression cases indicated a higher likelihood for disease events (HR=3.323, 95% CI 1.022-10.808; P=0.046; Table 2) in univariate analysis and exhibited a similar trend upon multivariate analysis (HR=2.792, 95% CI 0.838-9.300; P=0.094; Table 2). Taken together, our findings suggest that PROCR expression level can serve as a valuable prognostic marker for TNBC patients.

TABLE 2
Univariate and Multivariate survival analysis of factors
associated with disease-free survival in TNBC patients cohort

| | Univariate Analysis | | Multivariate Analysis | |
|---|---|---|---|---|
| | | P | | P |
| | HR (95% CI) | value | HR (95% CI) | value |
| Age | 1.271 (0.675-2.394) | 0.458 | | |
| Menopausal | 1.829 (0.959-3.488) | 0.067 | | |
| status | | | | |
| Tumor size | 1.682 (0.999-2.831) | 0.050 | 1.221 (0.674-2.211) | 0.510 |
| Lymph node | 2.464 (1.312-4.629) | 0.005 | 1.580 (0.730-3.421) | 0.245 |
| status | | | | |
| Stage | 2.184 (1.320-3.611) | 0.002 | 1.754 (0.921-3.340) | 0.087 |
| Histological | 1.012 (0.675-1.519) | 0.953 | | |
| grade | | | | |
| PROCR | 3.323 (1.022-10.808) | 0.046 | 2.792 (0.838-9.300) | 0.094 |

Abbreviations:
PROCR, protein C receptor;
TNBC, triple negative breast cancer

Inhibition of Procr Suppressed TNBC Formation

Next we investigate the significance of PROCR in human breast cancer formation. The expression level of PROCR was profiled in various breast cancer cell lines by qPCR analysis. PROCR was highly expressed in TNBC cell lines (MBA-MD-231, 2M4 and HS578T) compared to a ER+/PR+ line (MCF-7) and a Her2+ lines (SK-BR-3) (FIG. 18a). These are consistent with the results seen in patient tissue samples. We subsequently used MDA-MB-231 cells to investigate the impact of PROCR on tumor formation. ShRNA targeting PROCR (sh-PROCR) was generated (FIG. 19b). MDA-MB-231 cells were virally infected by Sh-PROCR and the knockdown efficacy in these cells was validated by Western analyses (FIG. 18b). In culture, the spindle-shaped morphology of the cell was altered to spherical looking (FIG. 18c), accompanied with reduced proliferation (FIG. 21a). Same morphological change was observed in the presence of the soluble PROCR (sPROCR, extracellular domain of PROCR), suggesting that the extracellular domain of PROCR facilitating ligand binding is important for its function in MDA-MB-231 cells (FIG. 22a, 22b). Protein C (PROC) is the established ligand in endothelial cells for anti-coagulation, anti-inflammation and cytoprotective activities of PROCR¹¹⁻¹⁶. To address the probability that the same ligand binds to PROCR in mammary epithelial cells, we generated the kinase dead form of PROC (PROC-KD). Addition of PROC-KD induced similar morphological changes in MDA-MB-231 cells (FIG. 22c), suggesting that PROC is the ligand for PROCR in breast cancer cells. Next, we examined the significance of PROCR in xenograft experiments. Knockdown of PROCR in MDA-MB-231 cells markedly delayed their tumor formation and attenuated tumor growth, while cells infected with scramble control potently formed tumors (FIG. 18d, 18e). Infection of Sh-PROCR was ineffective in influencing the MCF-7 tumor growth (FIG. 23a-c), indicating that the PROCR attenuation strategy is specific to TNBC cells.

To further evaluate the impact of PROCR in TNBC tumor growth, we employed three TNBC patient-derived xenografts (PDXs). Immunohistochemistry on PDX tumors indicates strong expression of PROCR in all three PDX tumors (FIG. 18f, FIG. 24). The knockdown efficacies of lentiviral Sh-PROCR in these PDX lines were validated by Western analyses (FIG. 18g and FIG. 25a, 25d). We found that knockdown of PROCR in all three PDX tumors significantly reduced tumor formation in xenograft experiments compared with the scramble control (FIG. 18h, 18i and FIG. 25). Taken together, our data suggest that the inhibition of PROCR can potently suppress human TNBC formation.

Next, we evaluate the therapeutic benefit of targeting PROCR using a more clinically applicable approach. We first tested a commercially available antibody, RCR-252. Interestingly, RCR-252 was able to detect PROCR overexpression in Western blot (FIG. 27b), but was not able to detect PROCR in immunohistochemistry assay in patient samples (FIG. 27c). Importantly, RCR-252 blocked the binding of PROCR and PROC (FIG. 27a) and changed the morphology of TNBC cells in culture (FIG. 27d). Injection of RCR-252 into Patient Derived Xenograft (PDX) models inhibited tumor growth (FIGS. 27e).

Another neutralizing antibody targeting the extracellular domain of PROCR was identified. This neutralizing antibody interfered the binding of PROCR with its ligand PROC, while the control non-neutralizing antibody could not (FIG. 18j). In MDA-MB-231 cell culture, addition of the neutralizing antibody transformed the spindle-shaped cells into spherical morphology, phenocoping PROCR shRNA treatment (FIG. 18k). Next, the anti-tumor activity of the neutralizing antibody was tested in PDXs. Recipient mice with engrafted PDX were treated with the antibody every two days for 5 times. Mice treated with control non-neutralizing antibody developed tumors that grow continuously, whereas treatment with the neutralizing antibody significantly suppressed tumor growth (FIG. 181). Consistent results were observed in another PDX model (PDX-2) (FIG. 26). These data reinforce our notion that functional inhibition of PROCR is able to suppress human TNBC formation.

Understanding the basic biology is a must for developing novel treatments for cancers. The cell of origin of TICs in different subtypes of breast cancer remains unclear despite some recent advance¹⁷⁻¹⁹. Gene expression profiling of Procr+ MaSCs has suggested similarities in normal and malignant stem cells³. In this study, our data indicate that Procr+ cells are indeed the TICs in MMTV-Wnt1 mouse tumor clustered close to human TNBC tumor, indicating that MaSCs are the cell of origin in this tumor subtype. Furthermore, PROCR is highly expressed in human TNBC tumors and correlated with poor patient survival. We establish that inhibition of PROCR defeats the tumorigenicity and progression of TNBC subtype. In conclusion, TNBC is an aggressive form of breast cancer with few available treatment options^{4,20}. Our study suggests that PROCR serves as a promising target for therapeutic intervention.

Methods

Experimental Animals. We generated the targeting construct of Procr^{flox} depicted in FIG. 20a. A loxP site was inserted upstream of exon 2, and an frt-flanked PGK-neo cassette followed by a second loxP site was inserted downstream of exon 4 of Procr gene. After genotyping described in FIG. 20b, Procr^{flox} mice were breed with a germline Flpase strain (Ella-Cre) to remove the frt-flanked neomycin selection cassette. Procr^{CreERT2-IRES-tdTomato} mouse was describe in a previous study³. For inducing Procr knockout, mice received intraperitoneal injection of 4 mg/25 g body weight of Tamoxifen (TAM, Sigma-Aldrich) diluted in sunflower oil every other day for a total of three times. MMTV-Wnt1, MMTV-Neu, MMTV-PyVT, MMTV-Cre, Brca1^{flox}, p53⁻ and Nude strain were used. Experimental procedures were approved by Animal Care and Use Committee of Shanghai Institute of Biochemistry and Cell Biology, Chinese Academy of Sciences.

Cell lines and cell culture. The MCF7, SK-BR-3, MDA-MB-231 (ATCC® Catalog No. HTB-26™), 2M4 and Hs-578T human breast cancer cell lines and the HEK293T cell line were obtained from the Shanghai Cell Bank Type Culture Collection Committee and maintained in complete growth medium as recommended by the distributor.

Antibodies. Antibodies used were: mouse anti human PROCR RCR-252 (1:300, Abcam Catalog No. ab81712), rabbit anti human PROCR (1:200, Novus), rat anti mouse K8 (1:250, Developmental hybridoma bank), rabbit anti mouse K14 (1:1000, Covance), rabbit anti Vimentin (1:50, Cell Signaling Technology), mouse anti ER (1:50, DAKO), mouse anti PR (1:50 DAKO), rabbit anti HER2 (1:50, Proteintech).

Primary Cell Preparation. Mammary glands from 8- to 12-week-old virgin or otherwise specified stage of female mice were isolated. The minced tissue was placed in culture medium (RPMI 1640 with 25 mM HEPES, 5% fetal bovine serum, 1% PSQ (Penicillin-Streptomycin-Glutamine), 300U ml⁻¹ Collagenase III [Worthington]) and digested for 2 hrs at 37° C. After lysis of the red blood cells in NH₄Cl, a single-cell suspension was obtained by sequential incubation with 0.25% trypsin-EDTA at 37° C. for 5 min and 0.1 mg/ml DNase I (Sigma) for 5 mins with gentle pipetting, followed by filtration through 70 um cell strainers.

Cell Labeling, Flow Cytometry. The following antibodies in 1:200 dilutions were used: biotinylated and FITC conjugated CD31, CD45, TER119 (BD PharMingen), CD24-PE/cy7, CD29-APC (Biolegend), Procr-PE (BD PharMingen), Streptavidin-V450, and Streptavidin-FITC (BD PharMingen). Antibody incubation was performed on ice for 15 min in HBSS with 10% fetal bovine serum. All sortings were performed using a FCASJazz (Becton Dickinson). The purity of sorted population was routinely checked and ensured to be more than 95%.

In Vitro Colony Formation Assay. FACS-sorted cells were resuspended at a density of 4×10⁵ cells ml⁻1 in chilled 100% growth factor reduced Matrigel (BD Bioscience), and the mixture was allowed to polymerized before covering with culture medium (DMEM/F12, ITS (1:100, Sigma) and 50 ng ml⁻¹ EGF. Culture medium was changed every 24 hrs. Primary colony numbers were scored after 6-7 days in culture. The colonies were mostly spherical. In cases that colonies were oval, the long axis was measured.

Immunohistochemistry. Whole mount staining was performed by fixation of the mammary gland in 4% paraformaldehyde for 1 hr and staining with Carmine overnight. Tissue paraffin sections were incubated with primary antibodies at 4° C. overnight, followed by washes, incubation with secondary antibodies for 2 hrs at 25° C., and counterstaining with DAPI (Vector Laboratories). For all of the immunoflourescence staining at least 3 independent experiments were conducted. Representative images are shown in the figures.

Overexpression and shRNA construct. Expression constructs for sPROCR (1-214 aa, extracellular domain) and Protein C (1-252 aa, a truncation of the kinase domain) were made using pCMV-Fc vector (addgene). Lentiviral expression constructs for mProcr and hPROCR overexpression were made using pHIV-zsgreen vectors carrying FLAG tag at the N terminus (addgene). The shRNA targeting mProcr or hPROCR sequences were constructed in lentivirus-based pLKO.1-EGFP constructs (addgene). The efficacy was individual shRNA was validated by Western blotting or qPCR. The sequences for mProcr-shRNA-1, mProcr-shRNA-2, hPROCR-shRNA-1 and hPROCR-shRNA-3 were 5′TTGTGTGGAGTTCCTGGAGAA3′ (SEQ ID NO.:19), 5′TCGGTATGAACTGCAGGAATT 3′(SEQ ID NO.:20), 5′GCAGCAGCTCAATGCCTACAA 3′(SEQ ID NO.:21) and 5′GCAGCAGCTCAATGCCTACAA3′ (SEQ ID NO.:22), respectively. If not specified, sh-Procr represents mProcr-shRNA-1, while sh-PROCR represents hPROCR-shRNA-1.

ELISA. Purified Protein C (100 ul, 0.2 ug/ml) was pre-coated to the bottom of a 96-well plate at 4C overnight. The wells were washed with PBS containing 0.5% Tween-20 and blocked with 1% BSA. A mixture of purified sPROCR (100 ul, 3 ug/ml) and the competing antibody or control antibody (in limiting dilution) were into the wells and incubated for 2 h at 37C. The bound sPROCR was detected after subsequent incubation with a biotin conjugated PROCR primary antibody (R&D Systems) for 1.5 hours and Streptavidin-HRP secondary antibody (R&D Systems) for 30 minutes. After HRP color detection, the absorbance was determined with a microplate reader at 450 nm. Samples were done in triplicate.

In vitro MDA-MB231 Morphology assay. MDA-MB-231 cells infected with scramble or PROCR shRNA were plated at a low density (5×10⁴) onto coverslips in 12-well plate using complete culture medium. After 12 hrs when cells are adhered to the coverslip, the plate are washed with PBS followed by fixation with 4% PFA for 10 min. Cells on coverslips are stained with Vimentin and DAPI counterstain. To examine the effect of various protein and antibodies on MDA-MB-231 cell morphology, purified IgG control, sPROCR (6 ug/ml), Protein C-kinase dead (2 ug/ml), control non-neutralizing and neutralizing antibodies (50 ug/ml) were used when cells are plated.

Mammary fat pad xenograft and analysis. Sorted cells were resuspended in 50% Matrigel, PBS with 20% FBS, and 0.04% Trypan Blue (Sigma), and injected in 10 ul volumes into the fat pads of 8-week-old Nude female. For in vivo knockdown with ShRNA, MMTV-Wnt1 tumor cells were virally infected by scramble or Sh-Procr; MDA-MB-231, MCF-7 and PDXs were virally infected by scramble or Sh-PROCR. The infected cells were sorted based on the tagged GFP expression in the ShRNA vector and resuspended in the above condition for transplantation. 2×10³ sorted MMTV-Wnt1 cells were inoculated into each fat pad. 3×10⁵ sorted MDA-MB-231 or MCF-7 cells were injected into each fat pad. To support the growth of the estrogen-dependent MCF-7 tumor, a 0.05-mg 17β-estradiol 21-day release pellet (Innovative Research of America) was implanted under the neck subcutaneous skin of the mouse on the day of tumor implantation. For PDX tumor formation, 5×10⁵ sorted PDX cells were inoculated into each fat pad. Tumor diameters were serially measured with calipers, and mouse weight was determined 3 times weekly. Tumor volume (in mm³) was calculated by the following formula: volume=length×width²×0.52. Mouse weight was monitored closely. For tumor inhibition by antibody experiments, PROCR non-neutralizing (control) antibody and neutralizing antibody (100 ug/mouse) were intraperitoneal administered 2 times a week for a total of 5 times. 3-4 mice per experimental group were used in animal experiments. All animals were of the same age and sex at the time of mammary epithelial cell injection or tumor cell injection. No statistical method was used to pre-determine sample size. The experiments were not randomized. There was no blinded allocation during experiments and outcome assessment.

Patients and specimens. For the immunohistochemical analysis of PRCOR in breast tumor whole-sections, a total of 80 stage I to III primary breast cancer samples from females with invasive ductal carcinoma were randomly collected at the Department of Breast Surgery at the Fudan University Shanghai Cancer Center between August 2013 and March 2014. The clinical pathologic diagnosis of breast cancer cases was determined by pathologists in the Department of Pathology. In our study, ER, PR, and human epidermal growth factor receptor 2 (HER2) expression statuses were also determined by IHC staining. Most, but not all, patients with HER2 expression status (IHC, score ≥2) were subjected to florescence in situ hybridization (FISH) screening for HER2 gene amplification. The HER2 overexpression subgroup was defined as FISH positive or an IHC staining score ≥3. As a result, the breast cancer patients were classified into four molecular subtypes according to the ER, PR, and HER2 status, including luminal A subtype (ER+ and/or PR+, low Ki67), luminal B subtype (ER+ and/or PR+, high Ki67 or HER2+), HER2+ subtype (HER2+, ER− and PR−), and triple-negative subtype (ER−, PR−, and HER2−). Total 80 breast cancer samples (20 for each of subtypes) were obtained to examine the PROCR protein level by immunohistochemical analysis using breast tumor whole-sections. To evaluate the prognostic value of PROCR in a large breast cancer patient cohort, we used Tissue microarrays (TMAs) containing 450 pathologically proven breast cancer samples and 72 non-cancerous mammary controls to examine the PROCR expression level. The eligibility criteria of breast cancer samples have been described in a previous study²¹. Briefly, the breast cancer patients in this cohort fulfilled the following inclusion criteria: (i) female patients diagnosed with stage I to III primary breast cancer; (ii) patients with unilateral invasive ductal carcinoma (IDC); ductal carcinomas in situ were excluded; (iii) patients without any evidence of metastasis at diagnosis; (iv) patients underwent a mastectomy and axillary lymph node dissection or breast conservation surgery followed by adjuvant chemotherapy; the therapeutic regimen decisions were based on the Chinese Anti-Cancer Association guidelines for the diagnosis and treatment of breast cancer.

For tissue microarrays (TMAs), we used the complete random sampling method to collect 207 luminal-like subtype cases, 93 HER2-enriched subtype cases and 150 triple-negative subtype cases from 1,709 cases that met the eligibility criteria and were diagnosed as breast cancer at the Department of Breast Surgery in FDSCC between August 2001 and January 2008. In addition, as described preciously²¹, a total of 72 non-cancerous mammary tissues controls with pathologically confirmed benign mammary diseases were also collected from women who had come to the Outpatient Department at FDSCC for breast cancer screening during the period from January 2013 to February 2013. This study was approved by the institutional review board (IRB) of Fudan University Shanghai Cancer Center (FDSCC), and all participants provided informed consent to participate in this research.

Tissue microarray (TMA). TMAs were constructed using above 450 paraffin-embedded blocks of breast tumors and 72 blocks of non-cancerous mammary controls using a tissue micro arrayer (UNITMA Instruments, Seoul, Korea). The hematoxylin and eosin (HE)-stained slides from tumors were evaluated to identify representative tumor regions. TMAs were composed of two 1.0-mm tissue cores from different areas of the same tumor to compare staining patterns. TMA sections were subsequently dewaxed in xylene and rehydrated in ethanol for IHC staining.

Immunohistochemical (IHC) staining. Immunohistochemistry for PROCR were conducted as previous described [REF]. Briefly, IHC for PROCR was performed using anti-PROCR antibody (1:300, Abcam) and Goat Anti-mouse HRP (1:1000, Santa Cruz) as secondary antibody followed by color development (DAKO) before counterstaining with hematoxylin.

Evaluation of IHC variables in breast tumor whole-sections and in TMAs. In 80-cases breast tumor whole-sections, expression of PROCR were semiquantitatively classified according to the immunoreactive H-score (HS; range 0-300) which was calculated as the result of the intensity score (1, faint/week; 2, moderate; 3, strong) multiplied by the distribution score (between 1 [percentage] to 100 [percentage]).

In TMAs, a total of 450 IDC breast cancer cases and 72 non-cancerous mammary tissues were included. Of these cases, 7 breast cancer cases and 1 non-cancerous sample experienced duplicate tissue core loss after IHC staining. Thus, the remaining 443 cancerous and 71 non-cancerous mammary samples were included in the subsequent analysis. The duplicate tissue cores from each case were also stained and scored semi-quantitatively using the same H-score evaluating criteria in breast tumor whole-sections.

Subsequently, stratification scoring was conducted according to H-score as follow: HS<80, scored as 0; 80<HS<120, scored as 1; 120<HS<150, scored as 2, HS>200, scored as 3. If the score was equal to or greater than one, the tumor was considered to have high PROCR expression; otherwise, low PROCR expression was classified. Based on the evaluation standard, scoring was reviewed in parallel by D S Wang and F Qiao; both examiners were blinded to all clinical data.

Kaplan-Meier analysis using TMAs and Kaplan-Meier Plotter. In the above cohort in TMAs, the breast cancer patients were regularly followed, and the clinical outcome of 415 cases was obtained, with the last update occurring in October 2014. The follow-up period was defined as the time from surgery to the last observation for censored cases or relapse/death for complete observations. Disease-free survival (DFS) was defined as the time from the date of primary surgery to the date of relapse/breast cancer-specific death or October 2014. The categories analyzed for DFS were first recurrence of disease at a local, regional, or distant site and breast cancer-specific death. Patients with study end date and loss of follow-up were considered censored. Thus, these 415 cancerous cases were analyzed in the Kaplan-Meier analysis.

In addition, a large public clinical database (Kaplan-Meier Plotter) of breast cancer was used to explore the association between PROCR expression and clinical outcomes, with the following restricted condition: 1) 140 months of follow-up time, 2): select media cutoff, 3) cases with ER status. Primary purpose of the tool is a meta-analysis based in silico biomarker assessment. We evaluated the effects of PROCR expression on disease-free survivals (DFS) of 671 hormone receptor-negative patients and 1802 hormone receptor-positive patients with the latest version of this database (2014 version; www.kmplot.com/analysis/index.php?p=service).

Generation of patient-derived xenografts from human breast cancers. PDX Processing and Passaging. PDX lines were originally initiated by implantation of a fresh patient tumor fragment into the mammary fat pad of recipient SCID/Beige mice and were maintained by serial passage in vivo at intervals characteristic for each line, and in accordance with Institutional Animal Care and Use Committee requirements. PDX tumors were excised, minced, and incubated at 37° C. for 1-3 h in digestion media [DMEM, 2% (vol/vol) FCS, 1×Pen-Step, 10 mM Hepes] with DNase, collagenase, and hyaluronidase. The suspension was then triturated and passed over a 40-μm cell strainer. Blood cells were lysed with ACK lysis buffer (Life Technologies). Cells were washed with HF buffer (Hank's Balanced Salt Solution, 2% FCS, 10 mM Hepes) and subjected to density gradient centrifugation using Optiprep (Sigma) to remove dead cells.

Statistical Analysis. Student's t-test was performed and the P value was calculated in Prism on data represented by bar charts, which consisted of results from three independent experiments unless specified otherwise. For all experiments with error bars, the standard deviation (s.d.) was calculated to indicate the variation within each experiment.

### REFERENCE

1 Carey, L., Winer, E., Viale, G., Cameron, D. & Gianni, L. Triple-negative breast cancer: disease entity or title of convenience? Nature reviews. Clinical oncology 7, 683-692, doi:10.1038/nrclinonc.2010.154 (2010).
2 Foulkes, W. D., Smith, I. E. & Reis-Filho, J. S. Triple-negative breast cancer. The New England journal of medicine 363, 1938-1948, doi:10.1056/NEJMra1001389 (2010).
3 Wang, D. et al. Identification of multipotent mammary stem cells by protein C receptor expression. Nature 517, 81-84, doi:10.1038/nature13851 (2015).
4 Lehmann, B. D. et al. Identification of human triple-negative breast cancer subtypes and preclinical models for selection of targeted therapies. The Journal of clinical investigation 121, 2750-2767, doi:10.1172/JC145014 (2011).
5 Tsukamoto, A. S., Grosschedl, R., Guzman, R. C., Parslow, T. & Varmus, H. E. Expression of the int-1 gene in transgenic mice is associated with mammary gland hyperplasia and adenocarcinomas in male and female mice. Cell 55, 619-625 (1988).
6 Herschkowitz, J. I. et al. Identification of conserved gene expression features between murine mammary carcinoma models and human breast tumors. Genome Biol 8, doi:Artn R76 Doi 10.1186/Gb-2007-8-5-R76 (2007).
7 Guy, C. T. et al. Expression of the Neu Protooncogene in the Mammary Epithelium of Transgenic Mice Induces Metastatic Disease. P Natl Acad Sci USA 89, 10578-10582, doi:Doi 10.1073/Pnas.89.22.10578 (1992).
8 Guy, C. T., Cardiff, R. D. & Muller, W. J. Induction of mammary tumors by expression of polyomavirus middle T oncogene: a transgenic mouse model for metastatic disease. Molecular and cellular biology 12, 954-961 (1992).
9 Xu, X. et al. Conditional mutation of Brcal in mammary epithelial cells results in blunted ductal morphogenesis and tumour formation. Nature genetics 22, 37-43, doi:10.1038/8743 (1999).
10 Rakha, E. A., Reis-Filho, J. S. & Ellis, I. O. Basal-like breast cancer: a critical review. Journal of clinical oncology: official journal of the American Society of Clinical Oncology 26, 2568-2581, doi:10.1200/JCO.2007.13.1748 (2008).
11 Taylor, F. B., Jr., Peer, G. T., Lockhart, M. S., Ferrell, G. & Esmon, C. T. Endothelial cell protein C receptor plays an important role in protein C activation in vivo. Blood 97, 1685-1688 (2001).
12 Cheng, T. et al. Activated protein C blocks p53-mediated apoptosis in ischemic human brain endothelium and is neuroprotective. Nat Med 9, 338-342, doi:10.1038/nm826 (2003).
13 Bae, J. S., Yang, L., Manithody, C. & Rezaie, A. R. The ligand occupancy of endothelial protein

C receptor switches the protease-activated receptor 1-dependent signaling specificity of thrombin from a permeability-enhancing to a barrier-protective response in endothelial cells. Blood 110, 3909-3916, doi:10.1182/blood-2007-06-096651 (2007).
14 Riewald, M., Petrovan, R. J., Donner, A., Mueller, B. M. & Ruf, W. Activation of endothelial cell protease activated receptor 1 by the protein C pathway. Science 296, 1880-1882, doi:10.1126/science.1071699 (2002).
15 Taylor, F. B., Jr. et al. The endothelial cell protein C receptor aids in host defense against Escherichia coli sepsis. Blood 95, 1680-1686 (2000).
16 Esmon, C. T. The protein C pathway. Chest 124, 26S-32S (2003).
17 Visvader, J. E. Cells of origin in cancer. Nature 469, 314-322, doi:nature09781 [pii]10.1038/nature09781.
18 Molyneux, G. & Smalley, M. J. The cell of origin of BRCA1 mutation-associated breast cancer: a cautionary tale of gene expression profiling. Journal of mammary gland biology and neoplasia 16, 51-55, doi:10.1007/s10911-011-9202-8 (2011).
19 Lim, E. et al. Aberrant luminal progenitors as the candidate target population for basal tumor development in BRCA1 mutation carriers. Nat Med 15, 907-913, doi:10.1038/nm.2000 (2009).
20 Metzger-Filho, O. et al. Dissecting the heterogeneity of triple-negative breast cancer. Journal of clinical oncology: official journal of the American Society of Clinical Oncology 30, 1879-1887, doi:10.1200/JCO.2011.38.2010 (2012).
21 Ye, F. G. et al. Cytidine Deaminase Axis Modulated by miR-484 Differentially Regulates Cell Proliferation and Chemoresistance in Breast Cancer. Cancer research 75, 1504-1515, doi:10.1158/0008-5472.CAN-14-2341 (2015).

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications, patents and patent applications referenced in this specification are incorporated herein by reference in their entirety for all purposes to the same extent as if each individual publication, patent or patent application were specifically indicated to be so incorporated by reference.

1. A method for diagnosis and/or treatment of triple negative breast cancer (TNBC), comprising detecting Protein C receptor (PROCR) selected from Procr gene, Procr mRNA and/or PROCR protein.

2. The method of claim 1, wherein an elevated expression level compared to non-TNBC control indicates the presence of TNBC or a subgroup within TNBC.

3. The method of claim 2, wherein the elevated expression level is detected by an amount of PRO CR mRNA and/or protein.

4. The method of claim 3, wherein the amount of PROCR mRNA and/or protein is more than 50%, more than 100% or more than 200% or higher than the non-TNBC control.

5. The method of claim 1, wherein decreasing PROCR level and/or activity provides TNBC or the subgroup treatment.

6. The method of claim 5, wherein said decreasing PROCR level and/or activity comprises one or more of: inhibiting Procr gene and/or mRNA stability and/or expression, reducing PROCR protein and/or neutralizing PROCR protein activity.

7. The method of claim 5, wherein the TNBC treatment comprises one or more of: (i) RCR-252 antibody, or antigen binding fragment thereof; (ii) an isolated anti-PROCR antibody or antigen binding fragment thereof wherein the antibody cross-competes for binding to PROCR with RCR-252; (iii) soluble PROCR fragment preferably comprising amino acids 18-210 of SEQ ID NO: 2; (iv) the interfering RNA designed to target Procr mRNA, and (v) CRISPR/Cas9 designed to target Procr gene.

8. (canceled)

9. (canceled)

10. A kit for diagnosing TNBC, comprising one or more of: (a) primers and/or probes designed to detect Procr mRNA; (b) RCR-252 antibody, or antigen binding fragment thereof; and (c) an isolated anti-PROCR antibody or antigen binding fragment thereof wherein the antibody cross-competes for binding to PROCR with RCR-252.

11. The kit of claim 10, further comprising instruction that when the amount of PRO CR mRNA and/or protein in a sample is more than 50%, more than 100% or more than 200% or higher than a non-TNBC control, then the sample is from TNBC.

12. (canceled)

13. (canceled)

14. A pharmaceutical composition for treating triple negative breast cancer, comprising a PROCR inhibitor and a pharmaceutically acceptable carrier, wherein the PROCR inhibitor is selected from the group consisting of: (i) RCR-252 antibody, or antigen binding fragment thereof; (ii) an isolated anti-PROCR antibody or antigen binding fragment thereof wherein the antibody cross-competes for binding to PROCR with RCR-252; (iii) interfering RNA designed to target Procr mRNA, and (iv) CRISPR/Cas9 designed to target Procr gene.

15-22. (canceled)
